(19) United States
(12) Patent Application Publication
(10) Pub. No.: US 2008/0171042 A1
(43) Pub. Date: Jul. 17, 2008

(54) Cd44 Variants As Therapeutic Targets

(75) Inventors: Kenneth A. Iczkowski, Gainesville, FL (US); Archangel Levi Omara-Opyene, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: University of Florida Research Foundation, Inc., Gainesville, FL (US)

(21) Appl. No.: 11/579,785

(22) PCT Filed: May 9, 2005
(86) PCT No.: PCT/US05/16216

§ 371 Date: Aug. 23, 2007

Related U.S. Application Data
(60) Provisional application No. 60/569,109, filed on May 7, 2004.

(51) Int. Cl. A61K 31/70; C07H 21/04; C12N 5/06; A61K 39/395; A61P 43/00

(52) U.S. Cl. 424/138.1; 514/44; 536/23.5; 435/375

(57) ABSTRACT

The present invention involves diagnostic and treatment methods for prostate cancer. We have shown that prostate cancer cells overexpress variant isoforms of CD44 (CD44v7-10). Overexpression is observed at the messenger RNA and protein levels. The present invention includes using diagnostic procedures such as RT-PCR and in situ hybridization to distinguish prostate cancer cells from benign prostate tissue. In addition, the present invention involves RNA interference (RNAi) targeted to a region of CD44v7-10 and to Muc18 as a treatment method for PC. We have shown that RNAi targeted to CD44v7-10 and to Muc 18 decreased invasiveness of two PC cell lines. Therapeutic methods of the present invention include gene therapy with RNAi, antisense, or ribozymes targeted against CD44v7-10 or Muc18 in cancer cells in vivo.

### CROSS REFERENCE TO RELATED APPLICATIONS

This application claims the benefit of U.S. Provisional Application 60/569,109, filed on May 7, 2004, which is hereby incorporated by reference in its entirety, including all tables, figures, references and nucleic acid and polynucleotide sequence listings.

### BACKGROUND OF THE INVENTION

Prostate carcinoma (PC) is the most common noncutaneous malignancy in men, affecting 10 million American men annually. One in six men develops prostate cancer in his lifetime, 189,000 annually (Jemal, A. et. al., 2002, CA Cancer J. Clin. 52:23-47). About 80% of cases are not clinically significant; they remain organ-confined and pose no threat during the patient's lifetime. Of the 20% of patients with clinically significant cancer, 10% suffer from advanced cancer at the time of diagnosis. The remaining 10% have early stage PC that will progress, but is potentially curable (Hall, R. R., 1996, Eur. Urol. 29 Supp 2:24-26).

Serum Prostate Specific Antigen (PSA) has revolutionized PC detection, but serum PSA is nonspecifically elevated by prostatitis (Goldman, H. B. et. al., 1997, World J. Urol. 15:257-261) and is expressed in ovarian cancer, lung cancer, myeloid leukemia cell lines, normal and cancerous pancreas, salivary gland, breast and the serum of women (Smith, M. R. et. al., 1995, Cancer Res. 55:2640-2644). The method of detecting serum PSA lacks sensitivity and specificity in the search for bloodborne or lymph nodal PC cells. Levels of tissue PSA detected by immunohistochemistry are lower in cancer than in benign prostate. Therefore, tissue PSA is not diagnostically useful nor is it a possible target of therapy. $1 billion is spent annually in the U.S. performing biopsies on men with benign prostates. Thus, new tissue and serum markers are needed for detection, prognosis, and therapeutic targets.

One such marker is cell adhesion protein CD44, which is one of several adhesion proteins with altered expression in PC. CD44 is a family of transmembrane glycoproteins involved in homotypic cell, cell-matrix, and cell-cytoskeletal interaction. The extracellular domain of CD44 binds numerous matrix substituents: hyaluronic acid, ezrin, radixin, moesin and merlin, heparin-affinity growth factors, vascular endothelial growth factor, p185HER2, epidermal growth factor, and hepatocyte growth factor. Its intracellular domain binds the cytoskeletal substituent ankyrin, thus determining cell and tissue architectural form (Bourguignon, L. Y. W. et. al., 1998, Front. Biosci. 3:D637-649; Welch, C. F. et. al., 1995, J. Cell. Physiol. 164:605-612). During metastasis, tumor cells detach from the primary site, migrate into the extracellular matrix, and invade blood and lymph vessels. Tumor outgrowth at the metastatic site requires attachment to the new extracellular matrix through adhesion proteins such as CD44.

The CD44 gene, which maps to chromosome 11, contains 20 exons spanning 60 kb, and can be subdivided into 5 structural domains. Ten exons (exons 1-5 and 16-20) constitute the ubiquitously expressed standard form of CD44 (CD44s). Ten additional exons in the extracellular portion of the protein can be alternatively spliced at the messenger RNA (mRNA) level, generating variant isoforms (CD44v) with over 1000 potential peptide domain combinations (FIG. 1). Theoretically, inclusion of all variant exons would yield a protein of molecular weight 230 kD, but most variant isoforms are less than 120 kD.

Longer, variant isoforms (CD44v1-10) include one or more of exons 6-15 spliced in, although in humans, exon 6 (v1) is not expressed. Some splice variants are expressed by normal epithelial cells in a tissue-specific fashion and CD44v10 is expressed by normal lymphocytes (Okamoto, I. et. al., 1998, J. Natl. Cancer Inst. 90:307-315). Cancers express novel variant isoforms, reflecting deregulated mRNA splicing.

Okamoto et al. (Okamoto, I. et. al., 2002, Am. J. Pathol. 160:441-447; Okamoto, I. et. al., 2001, J. Cell. Biol. 155:755-762; Murakami, D. et. al., 2003, Oncogene 22:1511-1516) have shown that, in several human tumors (exclusive of prostate cancer), cleavage products of 25-30 kD are detectable by Western blot using antibody against the cytoplasmic portion of CD44. The soluble portion of CD44 has been detected in serum as a 100-160-kD fragment using anti-CD44v monoclonal antibodies to extracellular portions of the molecule (Gansauge, F. et. al., 1997, Cancer 80:1733-1739). Western blot detection of CD44 isoforms shed into the circulation may serve as a diagnostic or prognostic test for malignancy (Taylor, D. D. et. al., 1996, J. Soc. Gynecol. Invest. 3:289-294). An enzyme-linked serum immunoassay (ELISA) may then be developed for sensitive, easier detection of the proteins.

CD44, along with KAI1 and MAP kinase 4, acts as a metastasis suppressor gene in prostate cancer (PC). Both prostate and bladder tumors lose protein antigen expression of CD44s (Iczkowski, K. A. et. al., 1997, J. Urol. Pathol. 6:119-129; Nagabhushan, M. et. al., 1996, Am. J. Clin. Pathol. 106:647-651) and CD44v6 (Iczkowski, K. A. et. al., 1997, J. Urol. Pathol. 6:119-129; De Marzo, A. M. et. al., 1998, Prostate 34:162-168) as the grade of the tumor increases, whereas amplification in CD44v6 is noted with metastatic phenotype of pancreatic cancer (Rall, C. J. and Rustgi, A. K., 1995, Cancer Res. 55:1831-1835) and increasing grade of breast cancer (Woodman, A. C. et. al., 1996, Am. J. Pathol. 149:1519-1530; Bourguignon, L. Y. et. al., 1999, Cell Motil. Cytoskeleton 43:269-287). Inclusion of single or contiguous variant exons has been described by RT-PCR and sequencing in many benign and cancer tissues (Okamoto, I. et. al., 1998, J. Natl. Cancer Inst. 90:307-315; Okamoto, I. et. al., 2002, Am. J. Pathol. 160:441-447; Rall, C. J. and Rustgi, A. K., 1995, Cancer Res. 55:1831-1835; Woodman, A. C. et. al., 1996, Am. J. Pathol. 149:1519-1530; Bourguignon, L. Y. et. al., 1999, Cell Motil. Cytoskeleton 43:269-287; Roca, X. et. al., 1998, Am. J. Pathol. 153:183-190; Franzmann, E. J. et. al., 2001, Otolaryngol. Head Neck Surg. 124:426-432; Terpe, H-J. et. al., 1996, Am. J. Pathol. 148:453-463; Christ, O. et. al., 2001, J. Leukoc. Biol. 69:343-352; Mortegani, M. P. et. al., 1999, Am. J. Pathol. 154:291-300; Miyake, H. et. al., 1998, Int. J. Cancer. 18:560-564; Yamaguchi, A. et. al., 1996, J. Clin. Oncol. 14:1122-1127).

We (Iczkowski, K. A. et. al., 1997, J. Urol. Pathol. 6:119-129), and subsequently others (Nagabhushan, M. et. al., 1996, Am. J. Clin. Pathol. 106:647-651; De Marzo, A. M. et. al., 1998, Prostate 34:162-168), showed that PC loses imrnunohistochemical expression of CD44s and some CD44v isoforms. CD44v6 was absent by immunohistochemistry in PC of all Gleason grades. Counter to this trend, PC reportedly overexpressed CD44 variant v7 compared with benign prostatic acini (Dhir, R. et. al., 1998, Mod. Pathol. 11:80A).

Most other cell adhesion molecules are downregulated or unchanged in PC. The exceptions are increased expression of Muc18 (CD146, or MelCAM) (Wu, G-J. et. al., 2001, Gene. 279:17-31; Wu, G-J. et. al., 2001, Prostate 48:305-315), N-cadherin and cadherin-11, although expression was consistently seen only in the highest Gleason score cancer (Tomita, K. et. al., 2000 Cancer Res. 60:3650-3654), and δ-catenin (Burger, M. J. et. al., 2002, Int. J. Cancer 100:228-237). Muc18 was originally found to be overexpressed on the surface of melanoma cells where it mediates their metastasis (Sers, C. et. al., 1994, Cancer Res. 54:5689-5694). Others have studied Muc18 in PC in some depth. Expression of Muc18 by prostate cancer cell lines (Wu, G-J. et. al., 2001, Gene. 279:17-31; Wu, G-J. et. al., 2001, Prostate 48:305-315) correlated with invasiveness and with in vivo metastasis in nude mice (Wu, G-J. et. al., 2001, Gene 279:17-31). Also, Muc18 immunohistochemical expression was increased in prostate cancer acini and their precursor lesion, prostatic intraepithelial neoplasia (PIN), in 37 cases (Wu, G-J. et. al., 2001, Prostate 48:305-315). Thus, we chose to study the effect of expression and silencing of Muc18.

Identification of specific variant CD44 isoforms overexpressed in prostate cancer cells will be useful in designing diagnostic methods for the detection and prognosis of prostate cancer. In addition, if a correlation can be established between increased expression of a specific protein in prostate cancer cells and increased growth/metastatic properties, therapeutic treatments can be targeted against the overexpressed protein.

### BRIEF SUMMARY OF THE INVENTION

The present invention involves diagnostic and treatment methods for prostate cancer. We have shown that prostate cancer cells overexpress variant isoforms of CD44 (CD44v7-10). Overexpression is observed at the messenger RNA and protein levels. The present invention includes using diagnostic procedures such as RT-PCR and in situ hybridization to distinguish prostate cancer cells from benign prostate tissue.

In addition, the present invention involves RNA interference (RNAi) targeted to a region of CD44v7-10 and to Muc18 as a treatment method for PC. We have shown that RNAi targeted to CD44v7-10 and to Muc 18 decreased invasiveness of two related PC cell lines, by 70% and 20% respectively. Therapeutic methods of the present invention include gene therapy with RNAi, antisense oligonucleotides, or ribozymes targeted against CD44v7-10 or Muc18 in cancer cells in vivo.

The present invention also includes the use of small molecular inhibitors designed to block the growth-promoting and metastatic activity of CD44v7-10 in the treatment of PC.

### BRIEF DESCRIPTION OF THE DRAWINGS

FIG. 1: An illustration of splice variant CD44 exons located in the extracellular domain. FIG. 1A: CD44v3 isoform is expressed in head and neck squamous cell carcinoma; FIG. 1B: CD44v3, v8-10 isoform is expressed in ductal breast carcinoma; FIG. 1C: CD44 v4-5 isoform is implicated in binding of tumor cells to hyaluronate; FIG. 1D: CD44v6 isoform is implicated in human carcinoma metastases and metastatic potential of pancreatic carcinoma and is also found in some breast carcinoma and in clear cell renal cell carcinoma; FIG. 1E: CD44v7 isoform is expressed on stromal cells allows homing of hematopoietic progenitor cells; FIG. 1F: CD44v8-10 (CD44E or epithelial form) is detectable in bladder cancer, some clear cell renal cell carcinoma, and colon cancer; FIG. 1G: CD44v7-10 is found in primary and metastatic prostatic adenocarcinoma.

FIG. 2: RT-PCR of CD44 variants from prostatic tumor and benign prostate tissue.

FIG. 3: RT-PCR and sequencing of CD44 variants from prostatic tumor tissue. FIG. 3A: CD44 variant products from RT-PCR amplification of prostatic tumor (T1, T2) and matched benign (B1, B2) tissues using primers E6 and P4; FIG. 3B: Control products from RT-PCR amplification of 18S ribosomal RNA from tumor and benign tissues (T1, T2, B1, B2); FIG. 3C: Sequence of the 608 base pair amplification product is identical to the published CD44v7-10 transcript that corresponds to variant exons 12-15; FIG. 3D: The 638 base pair amplification product is CD44v6-10, corresponding to variant exons 11-15; FIG. 3D: the 212 base pair amplification product is CD44v10, corresponding to variant exon 10.

FIG. 4: In situ hybridization for CD44v7 in benign acini shows little or no signal.

FIG. 5: In situ hybridization for CD44v7 in Gleason score 7 cancer shows strong, diffuse signal.

FIGS. 6A-B: Immunohistochemical staining for CD44v7 in prostate cancer tissue shows strong reactivity in acini with high-grade PIN in contrast to non-neoplastic acini.

FIG. 7: Western blot of five intermediate to high-grade tumors (T) and four benign specimens (B) using CD44 standard antibody. Reactivity at 85 kD corresponds to intact CD44 and is greatest in the benign specimens. Lower molecular weight bands of about 45 kD represent cleaved products present only in tumor, with the fourth tumor sample disclosing extra bands at 55 and 35 kD.

FIG. 8: Western blot of five intermediate to high-grade tumors (T) and four benign specimens (B) using CD44v6 antibody. Reactivity at 97 kD corresponds to intact CD44v6. Tumor tissues disclose slightly less reactivity for this band than benign tissue. Otherwise, no differences are seen between benign prostate and tumor specimens.

FIG. 9: Western blot with four intermediate to high-grade tumors (T) and five benign specimens (B) using CD44v7/8 antibody. All specimens show reactivity at 97 kD, consistent with intact CD44v7. In four tumor samples but not benign samples are prominent bands of reactivity at 45 kD. Tumor also demonstrates minor low-molecular weight signals ranging from 21 kD down to 6.5 kD.

FIG. 10: Western blot of five intermediate to high-grade tumors (T) and four benign specimens (B) using CD44v9 antibody. Benign tissues disclose more reactivity of the 85 kD band consistent with intact CD44v9 than tumor tissues. However, more prominent bands of reacitivity between 6.5 kD and 66 kD are evident in tumor tissue than in benign tissue. The 55 kD band is most prominent in the fourth and fifth tumor specimens.

FIG. 11A-C: Immunostaining of a tissue microarray for CD44v10 among prostatic tumors and benign prostate tissue showed preferential staining of tumor tissue. The same, significant trends have been observed for CD44v7/8 and CD44v9 using tissue microarrays. FIG. 11A: Percentage of CD44v10-immunoreactive cases out of 55 prostatic tumors compared to benign prostate tissue. FIG. 11B: CD44v10 immunostaining of benign prostate tissue; FIG. 11C: CD44v10 immunostaining of prostatic tumor tissue.

FIG. 12: Strategy for cloning RNAi plasmid constructs.

FIG. 13A-B: Western blot analysis of PC3M to detect expression of Muc18 in the absence and presence of RNAi targeted to Muc18 and CD44v9. FIG. 13A: Western blot detecting expression of Muc18 in PC3M cells transfected with i.) RNAi targeted to Muc18, ii.) RNAi targeted to Muc18 and CD44v9, and iii) no RNAi; FIG. 13B: Western blot detecting α-tubulin as a loading control for FIG. 13A.

FIG. 13C-D: Western blot analysis of Gₛα cells to detect expression of CD44v9 in the absence and presence of RNAi targeted to Muc18 and CD44v9. FIG. 13C: Western blot detecting expression of CD44v9 in GFP positive and GFP negative Gₛα cells transfected with i.) RNAi targeted to CD44v9, ii.) RNAi targeted to Muc18, iii.) no RNAi; FIG. 13D: Western blot detecting α-tubulin as a loading control for FIG. 13C.

FIG. 14A-D: Photomicrographs of Gₛα cells expressing GFP and RNAi targeted to CD44v9 and Matrigel following invasion by untreated Gₛα cells and Gₛα cells expressing RNAi targeted to CD44v9. FIG. 14A: Light microscopy of prostate Gₛα cells transfected with GFP and vector construct expressing RNAi targeted to CD44v9; FIG. 14B: Fluorescence microscopy of prostate Gₛα cells transfected with GFP and vector construct expressing RNAi targeted to CD44v9; FIG. 14C: Light microscopy of one focal plane of Matrigel stained after untreated Gₛα were allowed to invade; FIG. 14D: Light microscopy of one focal plane of Matrigel stained after GFP positive Gₛα cells (transfected with a vector construct expressing RNAi targeted to CD44v9) were allowed to invade.

FIG. 15A-D: Comparison of Matrigel invasion by prostate cancer cells untreated or expressing CD44v9 or Muc18 RNAi-causing DNA. FIG. 15A: Matrigel Invasion Index of PC3 and Gₛα cells following transfection with no construct or a construct expressing RNAi targeted to i.) CD44v9, ii.) Muc18, or iii.) both CD44v9 and Muc18; FIG. 15B: Percent of Matrigel invasion by GFP positive and GFP negative Gₛα cells transfected with vehicle or a construct expressing RNAi targeted to CD44v9 or Muc18.

FIG. 16: The nucleotide and amino acid sequence of the CD44 molecule (drawn from Screaton et al., Proc. Natl. Acad. Sci. USA, 1992, 89:12160-12164 [which is hereby incorporated by reference in its entirety with respect to the amino acid and nucleotide sequence of the CD44 molecule]). Junctional intron sequences are shown in lower case letters and the cDNA sequence is shown in upper case letters. The arrows indicate alternative splice donor and acceptor sites (exons 5 and 7 respectively).

FIG. 17. The volume and biologic potential (proliferation, apoptosis, angiogenic stimulation) of subcutaneous xenografts were altered by intratumoral RNAi injection against CD44v9. These trends have been consistently observed upon repeating the experiments, for a total of four treated and four control (sham-treated) animals. FIG. 17A. Mouse Gₛα-QL cell tumor. RNAi (Diamonds) caused more than 50% shrinkage of tumor between its initiation on day 28 and day 38, at which time the mouse was sacrificed. Tumor in control animal (Squares) shows no diminution. FIG. 17B. At day 28 of growth, upon initiation of RNAi injection therapy, tumor was 1.35 cm³. FIG. 17C. Mouse tumor on day 36, after 9 days of daily injection, was 0.715 cm³. FIGS. 17D-K. Immunostains. FIGS. 17D-E. Anti-CD44v9 (1:4 dilution of hybridoma supernatant). In the four untreated mice, tumor shows moderate diffuse reactivity with focal membranous accentuation (FIG. 17D). In the RNAi-treated (for 11 days) mouse, tumor reactivity is nearly absent although patchy foci are more reactive, perhaps reflecting uneven distribution of therapeutic DNA (FIG. 17E). FIGS. 17F-G. Anti-Proliferating Cell Nuclear Antigen (PCNA). In untreated mice, tumor shows nuclear reactivity of nearly 100% of nuclei (FIG. 17F). In the RNAi-treated mouse, the percent of reactive nuclei is moderately decreased, and average intensity is diminished (FIG. 17G). FIGS. 17H-I. Cleaved Caspase-3 (CC-3), an apoptosis marker. In untreated mice, tumor shows weak blush of CC-3 cytoplasmic reactivity (FIG. 17H). In treated mouse, tumor has moderate, diffuse cytoplasmic reactivity (FIG. 17I). FIGS. 17J-K. Vascular Endothelial Growth Factor (VEGF). FIG. 17J. In untreated mice, moderately strong diffuse cytoplasmic reactivity is seen for this stimulator of angiogenesis. FIG. 17K. In treated mouse, reactivity is diffusely diminished, although an occasional cell is more strongly reactive (inset).

FIG. 18. Injection of DNA to cause CD44 standard overexpression arrested the growth of Gₛα-QL tumor (Diamonds) as compared to untreated control (Squares).

### BRIEF DESCRIPTION OF THE SEQUENCES

SEQ ID NO: 1 [ATGGACAAGTTTTGGTGGCACGCAGCC] is the sequence of the P1 oligonucleotide primer located in exon 1 of CD44, upstream to the CD44 variant exon region.

SEQ ID NO: 2 [TTACACCCCAATCTTCATGTCCACATTC] is the sequence of the P2 oligonucleotide primer located in exon 20 of CD44, upstream to the CD44 variant exon region.

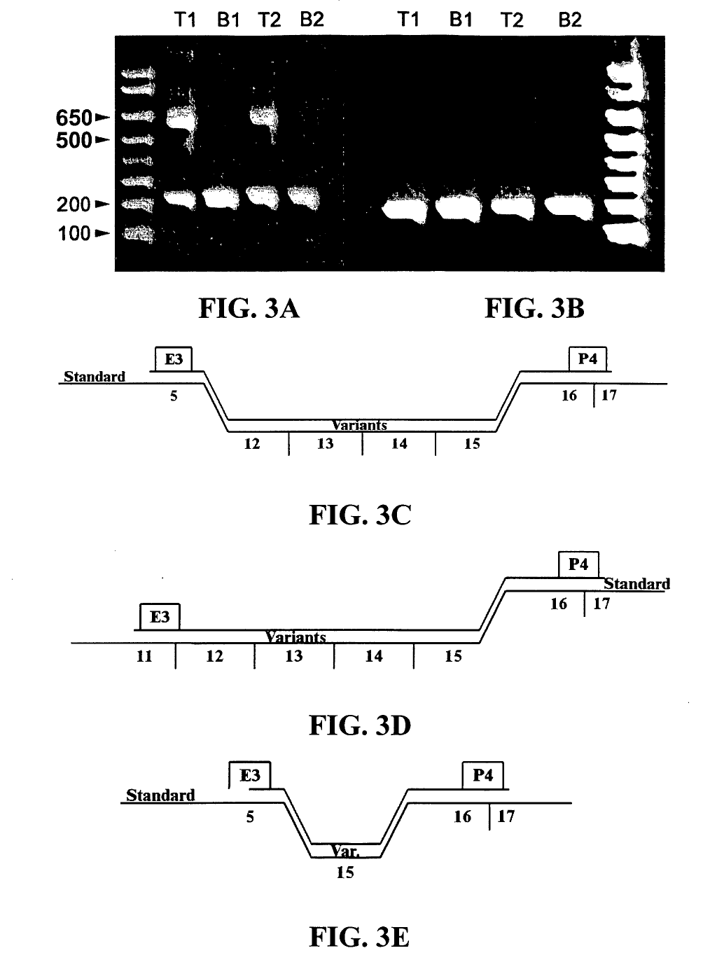

SEQ ID NO: 3 [AGCCCAGAGGACAGTTCCTGG (from FIG. 3)] is the sequence of the E3 oligonucleotide primer located upstream to the CD44 variant exon region.

SEQ ID NO: 4 [GATGCCAAGATGATCAGCCATTCTGGAA (from FIG. 3)] is the sequence of the P4 oligonucleotide primer located downstream to the CD44 variant exon region.

SEQ ID NO: 5 [actaatattgattccttcagATATGGACTCCAGTCATAGTACA ACGCTTCAGCCTACTGCAAATCCAAACACAGGTTTGGTGGAAGATTTGGACAGGA CAGGACCTCTTTCAATGACAACGCgtaagaataacgatgctcag] is a partial nucleotide sequence of the CD44v7 gene corresponding to intron 12, exon 12, and intron 13.

SEQ ID NO: 6 [TTCAGCCTACTGCAAATCCAAACACAGGTTTG] is the portion of the nucleotide sequence encoding CD44v7 used to create sense and antisense probes for in situ hybridization experiments.

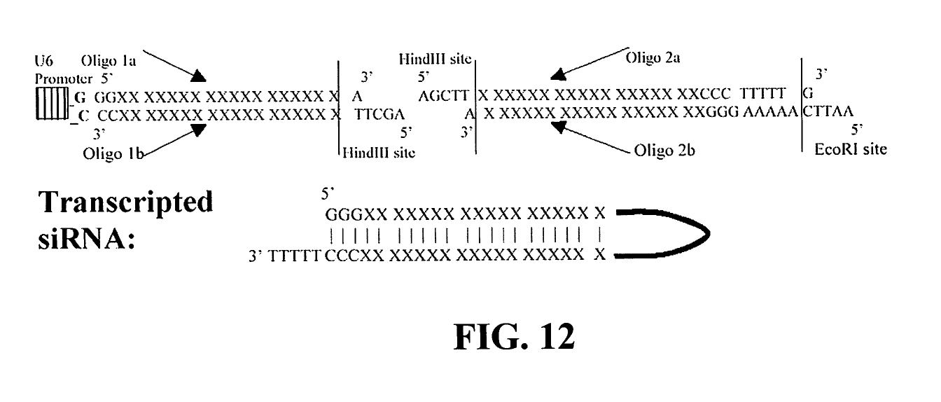

SEQ ID NO: 7 [GGTCCTTTGGAGTTACTGCAA] is the sequence of the oligonucleotide primer corresponding to Oligo 1a of FIG. 12 used to create CD44v9 dsRNA.

SEQ ID NO: 8 [AGCTTTGCAGTAACTCCAAAGGACC] is the sequence of the oligonucleotide primer corresponding to Oligo 1b of FIG. 12 used to create CD44v9 dsRNA.

SEQ ID NO: 9 [AGCTTTGCAGTAACTCCAAAGGACCC] is the sequence of the oligonucleotide primer corresponding to Oligo 2a of FIG. 12 used to create CD44v9 dsRNA.

SEQ ID NO: 10 [GGGTCCTTTGGAGTTACTGCAA] is the sequence of the oligonucleotide primer corresponding to Oligo 2b of FIG. 12 used to create CD44v9 dsRNA.

SEQ ID NO: 11 [GGCAGCACAGCCCTTCTGAAA] is the sequence of the oligonucleotide primer corresponding to Oligo 1a of FIG. 12 used to create Muc18 dsRNA.

SEQ ID NO: 12 [AGCTTTTCAGAAGGGCTGTGCTGCC] is the sequence of the oligonucleotide primer corresponding to Oligo 1b of FIG. 12 used to create Muc18 dsRNA.

SEQ ID NO: 13 [AGCTTTTCAGAAGGGCTGTGCTGCCCTTTTTG] is the sequence of the oligonucleotide primer corresponding to Oligo 2a of FIG. 12 used to create Muc18 dsRNA.

SEQ ID NO: 14 [AATTCTTTTTGGGCAGCACAGCCCTTCTGAAA] is the sequence of the oligonucleotide primer corresponding to Oligo 2b used to create Muc18 dsRNA.

SEQ ID NOs: 15-18 are the sequences of exons 12, 13, 14, and 15 (respectively) of CD44v7-10. SEQ ID NO: 19 represents joined exons 12-15.

SEQ ID NO: 20 is an alternative sequence that can be used for the production of RNAi or antisense DNA for use in the methods of the subject invention. [SEQ ID NO: 20 AGCCCAGAGGACAGTTCCTGGATCACCGACAGCACAGACAGAATCCCTGCTACCAAT ATGGACTCCAGTCATAGTATAACGCTTCAGCCTACTGCAAATCCAAACACAGGTTTG GTGGAAGATTTGGACAGGACAGGACCTCTTTCAATGACAACGCAGCAGAGTAATTCT CAGAGCTTCTCTACATCACATGAAGGCTTGGAAGAAGATAAAGACCATCCAACAACT TCTACTCTGACATTAAGCAATAGGAATGATGTCACAGGTGGAAGAAGAGACCCAAAT CATTCTGAAGGCTCAACTACTTTACTGGAAGGTTATACCTCTCATTACCCACACACGA AGGAAAGCAGGACCTTCATCCCAGTGACCTCAGCTAAGACTGGGTCCTTTGGAGTTA CTGCAGTTACTGTTGGAGATTCCAACTCTAATGTCAATCGTTCCTTATCAGGAGACCA AGACACATTCCACCCCAGTGGGGGGTCCCATACCACTCATGGATCTGAATCAGATGG ACACTCACATGGGAGTCAAGAAGGTGGAGCAAACACAACCTCTGGTCCTATAAGGAC ACCCCAAATTCCAGAATGGCTGATCATCTTGGCATC].

### BRIEF DESCRIPTION OF THE TABLES

Table I: Ratio of heavier CD44 mRNA bands to CD44s band at 482 bp after reverse transcriptase-PCR and agarose gel electrophoresis of CD44 mRNA isolated from cancer and benign tissue. Ratios listed in bold are ratios ≧0.39 in bands of 900 base pairs and above.

Table II: In situ hybridization of prostate cancer for CD44v7 (exon 12) including the mean signal intensity. Staining was evaluated as: 1+: heterogeneous expression in 5-20% of cells; 2+: expression in 20-70% of cells; 3+: strong expression in over 70% of cells.

Table III: Immunohistochemical Staining for CD44 variant 7/8 from 80 Prostate Cancer Cases.

Table IV: Nucleic acid sequences for exons 12, 13, 14 and 15 of the CD44 molecules. These exons, when joined together as exons 12-13-14-15 form the CD44v7-10 variant. Accession numbers for these sequences are also provided in the table.

### DETAILED DISCLOSURE OF THE INVENTION

The terms “comprising”, “consisting of” and “consisting essentially of” are defined according to their standard meaning. The terms may be substituted for one another throughout the instant application in order to attach the specific meaning associated with each term. The phrases “isolated” or “biologically pure” refer to material that is substantially or essentially free from components which normally accompany the material as it is found in its native state. Thus, isolated nucleic acids or polynucleotides in accordance with the invention preferably do not contain materials normally associated with the polynucleotides in their in situ environment.

“Nucleotide sequence”, “polynucleotide” or “nucleic acid” can be used interchangeably and are understood to mean, according to the present invention, either a double-stranded DNA, a single-stranded DNA or products of transcription of the said DNAs (e.g., RNA molecules). It should also be understood that the present invention does not relate to genomic polynucleotide sequences in their natural environment or natural state. The nucleic acid, polynucleotide, or nucleotide sequences of the invention can be isolated, purified (or partially purified), by separation methods including, but not limited to, ion-exchange chromatography, molecular size exclusion chromatography, or by genetic engineering methods such as amplification, subtractive hybridization, cloning, subcloning or chemical synthesis, or combinations of these genetic engineering methods.

A “complementary” polynucleotide sequence, as used herein, generally refers to a sequence arising from the hydrogen bonding between a particular purine and a particular pyrimidine in double-stranded nucleic acid molecules (DNA-DNA, DNA-RNA, or RNA-RNA). The major specific pairings are guanine with cytosine and adenine with thymine or uracil. A “complementary” polynucleotide sequence may also be referred to as an “antisense” polynucleotide sequence or an “antisense sequence”.

The subject invention provides for diagnostic assays based upon Western blot formats or standard immunoassays known to the skilled artisan for the detection of CD44v7-10 (or epitopes thereof). Antibodies for the various CD44 variants (e.g, CD44v7, CD44v8, CD44v7/8, CD44v9, CD44v10, or CD44v7-10) can be obtained from commercial sources. For example, antibody-based assays such as enzyme linked immunosorbent assays (ELISAs), radioimmunoassays (RIAs), imrnunohistochemical staining of tissues, lateral flow assays, reversible flow chromatographic binding assay (see, for example, U.S. Pat. No. 5,726,010, which is hereby incorporated by reference in its entirety), immunochromatographic strip assays, automated flow assays, and assays utilizing peptide- or antibody-containing biosensors may be employed for the detection of CD44 variants. The assays and methods for conducting the assays are well-known in the art and the methods may test biological samples (e.g., serum, plasma, tissue samples or blood) qualitatively (presence or absence of polypeptide) or quantitatively (comparison of a sample against a standard curve prepared using a polypeptide of the subject invention) for the presence of a particular CD44 variant.

Thus, the subject invention provides a method of detecting CD44 variants comprising contacting a biological sample with an antibody that specifically binds to epitopes onCD44v7-10 polypeptides (preferably to epitopes found in the region of v7-10) and detecting the presence of an antibody-antigen complex. The antibody-based assays can be considered to be of four types: direct binding assays, sandwich assays, competition assays, and displacement assays. In a direct binding assay, either the antibody or antigen is labeled, and there is a means of measuring the number of complexes formed. In a sandwich assay, the formation of a complex of at least three components (e.g., antibody-antigen-antibody) is measured. In a competition assay, labeled antigen and unlabelled antigen compete for binding to the antibody, and either the bound or the free component is measured. In a displacement assay, the labeled antigen is pre-bound to the antibody, and a change in signal is measured as the unlabelled antigen displaces the bound, labeled antigen from the receptor. Labels suitable for use in these detection methodologies include, and are not limited to 1) radioactive labels, 2) enzyme labels, 3) chemiluminescent labels, 4) fluorescent labels, 5) magnetic labels, or other suitable labels, including those set forth below.

Lateral flow assays can be conducted according to the teachings of U.S. Pat. No. 5,712,170 and the references cited therein. U.S. Pat. No. 5,712,170 and the references cited therein are hereby incorporated by reference in their entireties. Displacement assays and flow immunosensors useful for carrying out displacement assays are described in: (1) Kusterbeck et al., “Antibody-Based Biosensor for Continuous Monitoring”, in Biosensor Technology, R. P. Buck et al, eds., Marcel Dekker, N.Y. pp. 345-350 (1990); Kusterbeck et al., “A Continuous Flow Immunoassay for Rapid and Sensitive Detection of Small Molecules”, Journal of Immunological Methods, vol. 135, pp. 191-197 (1990); Ligler et al., “Drug Detection Using the Flow Immunosensor”, in Biosensor Design and Application, J. Findley et al., eds., American Chemical Society Press, pp. 73-80 (1992); and Ogert et al., “Detection of Cocaine Using the Flow Immunosensor”, Analytical Letters, vol. 25, pp. 1999-2019 (1992), all of which are incorporated herein by reference in their entireties. Displacement assays and flow immunosensors are also described in U.S. Pat. No. 5,183,740, which is also incorporated herein by reference in its entirety. The displacement immunoassay, unlike most of the competitive immunoassays used to detect small molecules, can generate a positive signal with increasing antigen concentration.

In various other aspects of the invention, the presence of CD44v7-10 can also be determined by hybridization studies under high stringency, intermediate stringency, and/or low stringency. Various degrees of stringency of hybridization can be employed. The more severe the conditions, the greater the complementarity that is required for duplex formation. Severity of conditions can be controlled by temperature, probe concentration, probe length, ionic strength, time, and the like. Preferably, hybridization is conducted under low, intermediate, or high stringency conditions by techniques well known in the art, as described, for example, in Keller, G. H., M. M. Manak [1987] DNA Probes, Stockton Press, New York, N.Y., pp. 169-170.

For example, hybridization of immobilized DNA on Southern blots with ³²P-labeled gene-specific probes can be performed by standard methods (Maniatis et al. [1982] Molecular Cloning: A Laboratory Manual, Cold Spring Harbor Laboratory, New York). In general, hybridization and subsequent washes can be carried out under intermediate to high stringency conditions that allow for detection of target sequences with homology to the exemplified polynucleotide sequence. For double-stranded DNA gene probes, hybridization can be carried out overnight at 20-25° C. below the melting temperature (Tₘ) of the DNA hybrid in 6× SSPE, 5× Denhardt's solution, 0.1% SDS, 0.1 mg/ml denatured DNA. The melting temperature is described by the following formula (Beltz et al. [1983] Methods of Enzymology, R. Wu, L. Grossman and K. Moldave [eds.] Academic Press, New York 100:266-285).

Tm=81.5° C.+16.6 Log [Na⁺]+0.41(% G+C)−0.61(% formamide)−600/length of duplex in base pairs.

Washes are typically carried out as follows:
(1) twice at room temperature for 15 minutes in 1×SSPE, 0.1% SDS (low stringency wash);
(2) once at Tₘ−20° C. for 15 minutes in 0.2×SSPE, 0.1% SDS (intermediate stringency wash).

For oligonucleotide probes, hybridization can be carried out overnight at 10-20° C. below the melting temperature (Tₘ) of the hybrid in 6×SSPE, 5× Denhardt's solution, 0.1% SDS, 0.1 mg/ml denatured DNA. Tₘ for oligonucleotide probes can be determined by the following formula:

Tₘ(° C.)=2(number T/A base pairs)+4(number G/C base pairs) (Suggs et al. [1981] ICN-UCLA Symp. Dev. Biol. Using Purified Genes, D. D. Brown [ed.], Academic Press, New York, 23:683-693).

Washes can be carried out as follows:
(1) twice at room temperature for 15 minutes 1×SSPE, 0.1% SDS (low stringency wash);
2) once at the hybridization temperature for 15 minutes in 1×SSPE, 0.1% SDS (intermediate stringency wash).

In general, salt and/or temperature can be altered to change stringency. With a labeled DNA fragment >70 or so bases in length, the following conditions can be used:

| | |
|---|---|
| Low: | 1 or 2X SSPE, room temperature |
| Low: | 1 or 2X SSPE, 42° C. |
| Intermediate: | 0.2X or 1X SSPE, 65° C. |
| High: | 0.1X SSPE, 65° C. |

By way of another non-limiting example, procedures using conditions of high stringency can also be performed as follows: Pre-hybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C., the preferred hybridization temperature, in prehybridization mixture containing 100 μg/ml denatured salmon sperm DNA and 5-20×10⁶ cpm of ³²P-labeled probe. Alternatively, the hybridization step can be performed at 65° C. in the presence of SSC buffer, 1×SSC corresponding to 0.15M NaCl and 0.05 M Na citrate. Subsequently, filter washes can be done at 37° C. for 1 h in a solution containing 2×SSC, 0.01% PVP, 0.01% Ficoll, and 0.01% BSA, followed by a wash in 0.1×SSC at 50° C. for 45 min. Alternatively, filter washes can be performed in a solution containing 2×SSC and 0.1% SDS, or 0.5×SSC and 0.1% SDS, or 0.1×SSC and 0.1% SDS at 68° C. for 15 minute intervals. Following the wash steps, the hybridized probes are detectable by autoradiography. Other conditions of high stringency which may be used are well known in the art and as cited in Sambrook et al., 1989, Molecular Cloning, A Laboratory Manual, Second Edition, Cold Spring Harbor Press, N.Y., pp. 9.47-9.57; and Ausubel et al., 1989, Current Protocols in Molecular Biology, Green Publishing Associates and Wiley Interscience, N.Y. are incorporated herein in their entirety.

Another non-limiting example of procedures using conditions of intermediate stringency are as follows: Filters containing DNA are pre-hybridized, and then hybridized at a temperature of 60° C. in the presence of a 5×SSC buffer and labeled probe. Subsequently, filters washes are performed in a solution containing 2×SSC at 50° C. and the hybridized probes are detectable by autoradiography. Other conditions of intermediate stringency which may be used are well known in the art and as cited in Sambrook et al., 1989, Molecular Cloning, A Laboratory Manual, Second Edition, Cold Spring Harbor Press, N.Y., pp. 9.47-9.57; and Ausubel et al., 1989, Current Protocols in Molecular Biology, Green Publishing Associates and Wiley Interscience, N.Y. are incorporated herein in their entirety.

Duplex formation and stability depend on substantial complementarity between the two strands of a hybrid and, as noted above, a certain degree of mismatch can be tolerated. Therefore, the probe sequences of the subject invention include mutations (both single and multiple), deletions, insertions of the described sequences, and combinations thereof, wherein said mutations, insertions and deletions permit formation of stable hybrids with the target polynucleotide of interest. Mutations, insertions and deletions can be produced in a given polynucleotide sequence in many ways, and these methods are known to an ordinarily skilled artisan. Other methods may become known in the future.

The subject invention provides, in one embodiment, methods for the identification of the presence of nucleic acids encoding CD44v7-10 polypeptides comprising contacting a sample with a nucleic acid specific for one or more exons of said CD44 variants (e.g., CD44v7 (exon 12), CD44v8 (exon 13), CD44v9 (exon 14) or CD44v10 (exon 15). In a preferred embodiment, the polynucleotide is a probe that is, optionally, labeled and used in the detection system. Polynucleotide sequences (of at least 8 nuleotides) that can be used in this aspect of the invention include those that hybridize with exons 12, 13, 14, 15, or any linear combination thereof that encode CD44 variants. Polynucleotide sequences (illustrated in Table IV) encoding these exons are known in the art and have accession numbers as set forth in Table IV. In some aspects of the invention, the probes have a length ranging from eight nucleotides to the full length of a given exon of CD44. In other aspects of the invention, the probes can be at least 8 consecutive nucleotides spanning one or more consecutive exons of CD44 (e.g., selected from the group of exons consisting of 12, 13, 14, and 15). Thus, probes of the subject invention can comprise at least 8 consecutive nucleotides of exons encoding CD44 (the maximum length of such probes being the full length nucleotide sequence of exons 12, 13, 14, or 15). Thus, a probe according to the subject invention can comprise 8 to 619 consecutive nucleotides of exons 12, 13, 14, and 15 of CD44. The term “successive” can be interchanged with the term “consecutive” or the phrase “contiguous span”. In some embodiments, a polynucleotide fragment or probe may be referred to as “a contiguous span of at least X nucleotides”, wherein X is any integer value between 8 and 619.

Many methods for detection of nucleic acids exist and any suitable method for detection is encompassed by the instant invention. Typical assay formats utilizing nucleic acid hybridization includes, and are not limited to, 1) nuclear run-on assay, 2) slot blot assay, 3) northern blot assay (Alwine et. al., Proc. Natl. Acad Sci. 74:5350), 4) magnetic particle separation, 5) nucleic acid or DNA chips, 6) reverse Northern blot assay, 7) dot blot assay, 8) in situ hybridization, 9) RNase protection assay (Melton et. al., Nuc. Acids Res. 12:7035 and as described in the 1998 catalog of Ambion, Inc., Austin, Tex.), 10) ligase chain reaction, 11) polymerase chain reaction (PCR), 12) reverse transcriptase (RT)-PCR (Berchtold et. al., Nuc. Acids. Res. 17:453), 13) differential display RT-PCR (DDRT-PCR) or other suitable combinations of techniques and assays. Labels suitable for use in these detection methodologies include, and are not limited to 1) radioactive labels, 2) enzyme labels, 3) chemiluminescent labels, 4) fluorescent labels, 5) magnetic labels, or other suitable labels, including those set forth below. These methodologies and labels are well known in the art and widely available to the skilled artisan. Likewise, methods of incorporating labels into the nucleic acids are also well known to the skilled artisan.

Also provided by the subject invention are methods of treating prostate cancer comprising the administration of a composition comprising an antibody that specifically binds to a CD44 variant to an individual having prostate cancer. The antibody can be monoclonal or polyclonal in nature. Similarly, polyclonal anti-MUC18 antibody has been used to inhibit prostate cancer motility and invasiveness in vitro (Wu G-J et al. Gene, 2001, 279:17-31). Also included within the scope of a CD44 variant specific antibody are humanized, chimeric, single chain, or other recombinant antibodies known in the art. In this aspect of the invention, the antibody can be administered systemically (e.g., intravenously or intraarterially) or locally (e.g., via direct injection into a loci of prostate cancer via intratumoral or stereotactic injection). CD44 variants to which the antibodies can specifically bind include CD44v9 or any antibody against the CD44v7-10 region.

The subject method also provides method of treating PC, reducing the volume of a tumor, reducing the invasiveness of carcinoma cells, or inducing apoptosis of carcinoma cells comprising the administration of antisense DNA molecules or RNAi (referred to, interchangeably, as interfering RNA (RNAi), double stranded RNA (dsRNA), silencing RNA (siRNA), or short hairpin RNA (shRNA)) to an individual or methods of reducing the invasiveness of carcinoma cells expressing CD44v7-10 comprising the administration of a composition comprising antisense nucleic acids or RNAi to an individual. In these aspects of the invention, compositions comprising antisense nucleic acids or RNAi can be introduced into a loci containing prostate carcinoma cells via methods known to those skilled in the art (e.g., by stereotactic injection or other means of directly introducing such nucleic acids to a loci containing carcinoma cells). In various aspects of this embodiment of the invention, the antisense or RNAi is administered to a patient.

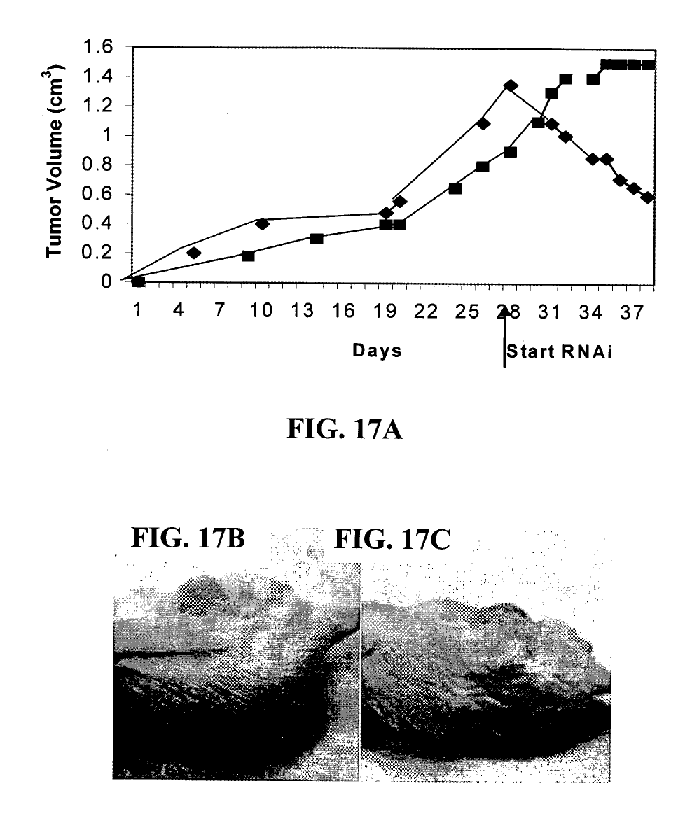

Compositions comprising antisense polynucleotides are to be understood as containing nucleic acid sequences that are complementary to the polynucleotides disclosed in SEQ ID NOs:15-19. Antisense polynucleotides can, optionally, including intron sequences bordering exons 12, 13, 14, or 15 (indicated as small letters in FIG. 16). Alternatively, antisense polynucleotides can include only the coding sequences for CD44v7, v8, v9 and/or v10 (e.g., CD44v7-10) indicated as capital letters in FIG. 16. While such polynucleotides can contain additional nucleic acid sequence derived from the CD44 molecule, a sufficient number of consecutive nucleotides of CD44 exons 12, 13, 14, and/or 15 must be present in said antisense polynucleotide to allow for the hybridization of the antisense sequence with its target sequence.

The invention also provides for compositions comprising dsRNA (RNAi, shRNA, or siRNA) to exons 12, 13, 14 and/or 15 of the CD44 molecule for use in the treatment regimens. dsRNA typically comprises a first polynucleotide sequence (a first nucleic acid strand) identical to a target gene (or fragment thereof) linked directly, or indirectly, to a second polynucleotide sequence (a second nucleic acid strand) complementary to the sequence of the target gene (or fragment thereof). In some embodiments, the first nucleic acid strand and second nucleic acid strand are fully complementary to one another over their full length. Other embodiments provided for mismatches between the first and second nucleic acid strands (e.g., 1, 2, 3, 4, 5, 6, 7, 8, 9, or 10 mismatches between the strands). The dsRNA may further comprise a chemical liner or a polynucleotide linker sequence of sufficient length to allow for the two polynucleotide sequences to fold over and hybridize to each other; however, a linker sequence is not necessary. The linker sequence is designed to separate the antisense and sense strands of RNAi significantly enough to limit the effects of steric hindrances and allow for the formation of dsRNA molecules. Linkers can be between 4 and 100 nucleotides, preferably between 4 and 50 nucleotides, and more preferably between 4 and 25 nucleotides in length. Chemical linkers suitable for use in the subject invention can be obtained from Pierce Biotechnology, Inc. (Rockford, Ill.).

dsRNA containing a nucleotide sequence identical to a fragment of the target gene is preferred for inhibition as RNAi; however, dsRNA sequences with insertions, deletions, and point mutations relative to the target sequence can also be used for inhibition. Sequence identity may optimized by sequence comparison and alignment algorithms known in the art and calculating the percent difference between the nucleotide sequences by, for example, the Smith-Waterman algorithm as implemented in the BESTFIT software program using default parameters (e.g., University of Wisconsin Genetic Computing Group). Alternatively, the duplex region of the RNA may be defined functionally as a nucleotide sequence that is capable of hybridizing with a fragment of the target gene transcript.

As disclosed herein, 100% sequence identity between the dsRNA and the target gene is not required to practice the present invention. Thus the invention has the advantage of being able to tolerate sequence variations that might be expected due to genetic mutation, polymorphisms, or evolutionary divergence. RNA may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands). The RNA strands may or may not be polyadenylated; the RNA strands may or may not be capable of being translated into a polypeptide by a cell's translational apparatus. RNA may be chemically or enzymatically synthesized by manual or automated reactions. If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

dsRNA can be targeted to an entire polynucleotide sequence of a CD44 exon (e.g., exon 12, 13, 14, or 15). Alternatively, the each strand (e.g., the first and second nucleic acid strand) of the dsRNA molecule can comprise at least about 15 consecutive nucleotides of exons 12, 13, 14, or 15 (with a maximum length equaling the number of nucleotides in each respective exon (as indicated in capital letters in FIG. 16)) or sequences complementary thereto. Other embodiments allow for dsRNA of at least 15 consecutive nucleotides spanning one or more consecutive exons of the CD44 molecules (e.g., exons 12-13, 13-14, 14-15, 12-13-14, 13-14-15, or 12-13-14-15). In such aspects of the invention, the each strand of a dsRNA molecule ranges from about 15 to 459 consecutive nucleotides of SEQ ID NO: 19, provided that the molecules span one or more exon selected from the group of exons consisting of 12, 13, 14, and 15 of CD44. Thus, one strand of a dsRNA molecule comprising a contiguous span of at least X consecutive nucleotides of SEQ ID NO: 19 can be used in the practice of the claimed invention, wherein X is any integer value between 15 and 459, provided that the contiguous span spans at least two of consecutive exons selected from 12, 13, 14, and 15.

In certain preferred embodiments, dsRNA according to the subject invention is composed of two complementary strands of nucleic acids, each strand of nucleic acids containing a contiguous span of Y nucleotides, of SEQ ID NOs: 15, 16, 17, 18, or 19, wherein Y is an integer selected from the group consisting of 15, 16, 17, 18, 19, 20, 21, 22, 23, 24, 25, 26, 27, 28, 29, 30, 31, 32, 33, 34, 35, 36, 37, 38, 39, 50, 41, 42, 43, 44, 45, 46, 47, 48, 49, and 50. The contiguous span of Y nucleotides can, optionally, span two consecutive exons of CD 44 selected from the group consisting of exons 12, 13, 14, and 15. Preferred RNAi molecules of the instant invention are highly homologous or identical to the polynucleotides of SEQ ID NOs: 15-19. The homology is preferably greater than 90% and is most preferably greater than 95%.

Thus, RNAi molecules of the subject invention are not limited to those that are targeted to the full-length polynucleotide or gene. The nematode gene product can be inhibited with an RNAi molecule that is targeted to a portion or fragment of the exemplified polynucleotides; high homology (90-95%) or identity is also preferred, but not necessarily essential, for such applications. If desired, RNAi molecules can be designed for target DNA sequences using commercially available software (e.g., siRNA DNA Designer 1.5, IRIS Genetics, Houston, Tex.).

TABLE IV

Nucleic Acid Coding Sequences for Exons 12-15 of CD44 (coding sequences
(exons) shown in all capital letters (see FIG. 16))

CD44 Exon 12 (142 Base Pairs) Accession # L05417

[SEQ ID NO: 15]

actaatattg attccttcag ATATGGACTC CAGTCATAGT ACAACGCTTC AGCCTACTGC AAATCCAAAC
ACAGGTTTGG TGGAAGATTT GGACAGGACA GGACCTCTTT CAATGACAAC GCgtaagaat aacgatgctc
ag
CD44 Exon 13 (130 Base Pairs) Accession # L05418

[SEQ ID NO: 16]

ttcattcctc attgaaacag AGCAGAGTAA TTCTCAGAGC TTCTCTACAT CACATGAAGG CTTGGAAGAA
GATAAAGACC ATCCAACAAC TTCTACTCTG ACATCAAGCA gtaaggatta taaaacctag
CD44 Exon 14 (244 Base Pairs) Accession # L05419

[SEQ ID NO: 17]

ctgattccac ctccacacag ATAGGAATGA TGTCACAGGT GGAAGAAGAG ACCCAAATCA TTCTGAAGGC
TCAACTACTT TACTGGAAGG TTATACCTCT CATTACCCAC ACACGAAGGA AAGCAGGACC TTCATCCCAG
TGACCTCAGC TAAGACTGGG TCCTTTGGAG TTACTGCAGT TACTGTTGGA GATTCCAACT CTAATGTCAA
TCGTTCCTTA TCAGgtaatt tggcatttat tatc
CD44 Exon 15 (103 base pairs) Accession # L05420

[SEQ ID NO: 18]

ttcctgattg ctcattacag GAGACCAAGA CACATTCCAC CCCAGTGGGG GGTCCCATAC CACTCATGGA
TCTGAATCAG ATGgtgagtt caaaactgct tta
CD44 Exons 12-15 (459 base pairs)

[SEQ ID NO: 19]

ATATGGACTC CAGTCATAGT ACAACGCTTC AGCCTACTGC AAATCCAAAC ACAGGTTTGG TGGAAGATTT
GGACAGGACA GGACCTCTTT CAATGACAAC GCAGCAGAGT AATTCTCAGA GCTTCTCTAC ATCACATGAA
GGCTTGGAAG AAGATAAAGA CCATCCAACA ACTTCTACTC TGACATCAAG CAATAGGAAT GATGTCACAG
GTGGAAGAAG AGACCCAAAT CATTCTGAAG GCTCAACTAC TTTACTGGAA GGTTATACCT CTCATTACCC
ACACACGAAG GAAAGCAGGA CCTTCATCCC AGTGACCTCA GCTAAGACTG GGTCCTTTGG AGTTACTGCA
GTTACTGTTG GAGATTCCAA CTCTAATGTC AATCGTTCCT TATCAGGAGA CCAAGACACA TTCCACCCCA
GTGGGGGGTC CCATACCACT CATGGATCTG AATCAGATG

The subject invention also provides methods of treating cancer, carcinomas, or tumors expressing CD44 variants comprising the overexpression of CD44s (the normal form of CD44) within cells of the cancer, carcinoma or tumor (“target cells”) and causing a reduction or arrest of tumor growth. This aspect of the invention can be practiced by the introduction of a vector comprising the CD44s molecule into a locus containing cancer, carcinoma or tumor cells. For example, the vector can be introduced by stereotactic injection (e.g., MRI guided or ultrasound guided stereotactic injection) or nucleic acids encoding CD44s can be introduced into target cells as discussed below. Any nucleic acid sequence encoding CD44s polypeptide can be used in the practice of this aspect of the invention. CD44s polypeptide can include or lack the signal sequence found in the unprocessed form of the polypeptide at the option of the practitioner. As indicated supra, CD44s is composed of exons 1-5 and 16-20.

Retrovirus, adeno-associated virus, and vectors such as pTRACER can be used for the delivery of CD44s encoding polynucleotides into target cells. These vectors can also be used for the in vivo introduction of CD44s into target cells for the overexpression of the polypeptide. To effect expression of the polynucleotides and polynucleotide constructs encoding CD44s, the constructs must be delivered into a cell. This delivery may be accomplished in vitro, as in laboratory procedures for transforming cell lines, or in vivo or ex vivo. One mechanism is viral infection where the expression construct is encapsulated in an infectious viral particle. The expression construct, preferably a recombinant viral vector as discussed herein, may transduce packaging cells through any means known in the art such as electroporation, liposomes, and CaPO₄ precipitation. The packaging cell generates infectious viral particles that include a polynucleotide encoding a CD44s polypeptide. Such viral particles then may be employed to transduce eukaryotic cells in vitro, ex vivo or in vivo. The transduced eukaryotic cells will overexpress CD44s in a target cell and viruses used in the present invention can be rendered replication deficient by deletion of one or more of all or a portion of the following genes: E1a, E1b, E3, E4, E2a, or L1 through L5 (U.S. Pat. No. 6,228,844, the disclosure of which is hereby incorporated by reference in its entirety).

Retrovirus vectors and adeno-associated virus vectors provide efficient delivery of CD44s into cells, and the transferred nucleic acids can be stably integrated into the chromosomal DNA of the target cell. A major prerequisite for the use of retroviruses is to ensure the safety of their use, particularly with regard to the possibility of the spread of wild-type virus in the cell population. Thus, recombinant retrovirus can be constructed in which part of the retroviral coding sequence (gag, pol, env) has been replaced by nucleic acids that render the retrovirus replication defective. The replication defective retrovirus is then packaged into virions which can be used to infect a target cell through the use of a helper virus by standard techniques.

Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include psiCrip, psiCre, psi2 and psiAm. Retroviruses have been used to introduce a variety of genes into many different cell types, including neural cells, epithelial cells, endothelial cells, lymphocytes, myoblasts, hepatocytes, bone marrow cells, in vitro and/or in vivo (see for example Eglitis et. al. (1985) Science 230:1395-1398; Danos and Mulligan (1988) Proc. Natl. Acad. Sci. USA 85:6460-6464; Wilson et. al. (1988) Proc. Natl. Acad. Sci. USA 85:3014-3018; Armentano et. al. (1990) Proc. Natl. Acad. Sci. USA 87:6141-6145; Huber et. al. (1991) Proc. Natl. Acad. Sci. USA 88:8039-8043; Ferry et. al. (1991) Proc. Natl. Acad. Sci. USA 88:8377-8381; Chowdhury et. al. (1991) Science 254:1802-1805; van Beusechem et. al. (1992) Proc. Natl. Acad. Sci. USA 89:7640-7644; Kay et. al. (1992) Human Gene Therapy 3:641-647; Dai et. al. (1992) Proc. Natl. Acad. Sci. USA 89:10892-10895; Hwu et. al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

The infection spectrum of retroviruses and retroviral-based vectors can be limited, by modifying the viral packaging proteins on the surface of the viral particle (see, for example PCT publications WO93/25234, WO94/06920, and WO94/11524). For instance, strategies for the modification of the infection spectrum of retroviral vectors include: coupling antibodies specific for cell surface antigens (e.g., cd44V9) to the viral env protein (Roux et. al. (1989) PNAS 86:9079-9083; Julan et. al. (1992) J. Gen Virol 73:3251-3255; and Goud et. al. (1983) Virology 163:251-254); or coupling cell surface ligands to the viral env proteins (Neda et. al. (1991) J Biol Chem 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector. Moreover, use of retroviral gene delivery can be further enhanced by the use of tissue- or cell-specific transcriptional regulatory sequences that control expression of the desired gene.

Another viral gene delivery system useful for the overexpression of CD44s in target cells utilizes adenovirus-derived vectors. The genome of an adenovirus can be manipulated to overexpress CD44s and inactivate the virus' ability to replicate in a normal lytic viral life cycle (see, for example, Berkner et. al. (1988) BioTechniques 6:616; Rosenfeld et. al. (1991) Science 252:431-434; and Rosenfeld et. al. (1992) Cell 68:143-155). Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types, including airway epithelium (Rosenfeld et. al. (1992) cited supra), endothelial cells (Lemarchand et al. (1992) Proc. Natl. Sci. USA 89:6482-6486), hepatocytes (Herz and Gerard (1993) Proc. Natl. Acad. Sci. USA 90:2812-2816) and muscle cells (Quantin et. al. (1992) Proc. Natl. Acad. Sci. USA 89:2581-2584). Furthermore, the virus particle is relatively stable and amenable to purification and concentration, and as above, can be modified so as to affect the spectrum of infectivity. Additionally, introduced adenoviral polynucleotides (and foreign polynucleotides contained therein) are not integrated into the genome of a target cell but remains episomal, thereby avoiding potential problems that can occur as a result of insertional mutagenesis in situations where introduced DNA becomes integrated into the target cell genome (e.g., retroviral DNA). Moreover, the carrying capacity of the adenoviral genome for foreign DNA is large (up to 8 kilobases) relative to other gene delivery vectors (Haj-Ahmand and Graham (1986) J. Virol. 57:267). Most replication-defective adenoviral vectors currently in use are deleted for all or parts of the viral E1 and E3 genes but retain as much as 80% of the adenoviral genetic material (see, e.g., Jones et. al. (1979) Cell 16:683; Berkner et. al, supra; and Graham et. al. in Methods in Molecular Biology, E. J. Murray, Ed. (Humana, Clifton, N.J., 1991) vol. 7. pp. 109-127). Expression of the CD44s polynucleotides can be under control of, for example, the E1A promoter, the major late promoter (MLP) and associated leader sequences, the E3 promoter, or exogenously added promoter sequences.

Yet another viral vector system useful for delivery of polynucleotides is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et. al., Curr. Topics in Micro. and Immunol. (1992) 158:97-129). It is also one of the few viruses that may integrate its nucleic acids into non-dividing cells, and exhibits a high frequency of stable integration (see for example Flotte et al., (1992) Am. J. Respir. Cell Mol. Biol. 7:349-356; Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et. al (1985) Mol. Cell. Biol. 5:3251-3260 can be used to introduce DNA into cells. A variety of nucleic acids have been introduced into different cell types using AAV vectors (see for example Hermonat et. al. (1984) Proc. Natl. Acad. Sci. USA 81:6466-6470; Tratschin et. at (1985) Mol. Cell. Biol. 4:2072-2081; Wondisford et. al (1988) Mol. Endocrinol. 2:32-39; Tratschin et. at (1984) J. Virol. 51:611-619; and Flotte et. al (1993) J. Biol. Chem. 268:3781-3790).

Other viral vector systems that may have application in gene therapy have been derived from herpes virus, vaccinia virus, and several RNA viruses. In particular, herpes virus vectors may provide a unique strategy for persistence of inserted gene expression in cells of the central nervous system and ocular tissue (Pepose et. al (1994) Invest Ophthalmol Vis Sci 35:2662-2666, the disclosure of which is hereby incorporated by reference in its entirety).

Several non-viral methods for the transfer of CD44s encoding polynucleotides into cultured mammalian cells are also contemplated by the present invention, and include, without being limited to, calcium phosphate precipitation [Graham et al., (1973) Virol. 52:456-457; Chen et al. (1987) Mol. Cell. Biol. 7:2745-2752]; DEAE-dextran [Gopal (1985) Mol. Cell. Biol., 5:1188-1190]; electroporation [Tur-Kaspa et al. (1986) Mol. Cell. Biol. 6:716-718; Potter et al., (1984) Proc. Natl. Acad. Sci. U.S.A. 81(22):7161-7165]; direct microinjection (Harland et al., (1985) J. Cell. Biol. 101:1094-1095); DNA-loaded liposomes [Nicolau et al., (1982) Biochim. Biophys. Acta. 721:185-190; Fraley et al., (1979) Proc. Natl. Acad. Sci. USA. 76:3348-3352]; and receptor-mediated transfection. [Wu and Wu (1987), J. Biol. Chem. 262:4429-4432; and Wu and Wu (1988), Biochemistry 27:887-892], which disclosures are hereby incorporated by reference in their entireties. Some of these techniques may be successfully adapted for in vivo or ex vivo use, as discussed herein.

Once the expression polynucleotide has been delivered into the target cell, it may be stably integrated into the genome of the recipient cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the target cell cycle.

One non-limiting embodiment for a method for delivering of CD44s and directing its overexpression in the target cell in vivo comprises the step of introducing a preparation comprising a physiologically acceptable carrier and a naked polynucleotide operatively coding for CD44s into the interstitial space of a tissue comprising the target cell (e.g., a tumor or locus of a cancer or carcinoma), whereby the naked polynucleotide is taken up into the interior of the cell and has a physiological effect. This is particularly applicable for transfer in vitro but it may be applied to in vivo as well. Compositions for use in vitro and in vivo comprising a “naked” polynucleotide are described in PCT application No. WO 90/11092 (Vical Inc.) and also in PCT application No. WO 95/11307 (Institut Pasteur, INSERM, Université d'Ottawa) as well as in the articles of Tascon et al. (1996), Nature Medicine. 2(8):888-892 and of Huygen et al., (1996) Nature Medicine. 2(8):893-898, the disclosures of which are each hereby incorporated by reference in their entireties. In still another embodiment of the invention, the transfer of a naked polynucleotide encoding CD44s into cells may be accomplished with particle bombardment (biolistic), said particles being DNA-coated microprojectiles accelerated to a high velocity allowing them to pierce cell membranes and enter cells without killing them, such as described by Klein et al., (1987) Nature 327:70-73, which disclosure is hereby incorporated by reference in its entirety. Liposomal preparations for use in the present invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner et. al., Proc. Nat. Acad. Sci. USA (1987) 84:7413-7416, which is herein incorporated by reference); mRNA (Malone et. al., Proc. Natl. Acad. Sci. USA (1989) 86:6077-6081, which is herein incorporated by reference); and purified transcription factors (Debs et al., J. Biol. Chem. (1990) 265:10189-10192, which is herein incorporated by reference), in functional form.

Cationic liposomes are readily available. For example, N[1-2,3-dioleyloxy)propyll-N,N,N-triethylammonium (DOTMA) liposomes are particularly useful and are available under the trademark Lipofectin, from GIBCO BRL, Grand Island, N.Y. (See, also, Feigner et. al., Proc. Nad Acad. Sci. USA (1987) 84:7413-7416, which is herein incorporated by reference). Other commercially available liposomes include transfectace (DDAB/DOPE) and DOTAP/DOPE (Boehringer). Similarly, anionic and neutral liposomes are readily available, such as from AvantiPolar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolarnine (DOPE), among others. These materials can also be mixed with the DOTMA and DOTAP starting materials in appropriate ratios. Methods for making liposomes using these materials are well known in the art. For example, commercially dioleoylphosphatidyl choline (DOPC), dioleoylphosphatidyl glycerol (DOPG), and dioleoylphosphatidyl ethanolarnine (DOPE) can be used in various combinations to make conventional liposomes, with or without the addition of cholesterol. The liposomes can comprise multilamellar vesicles (MLVs), small unilamellar vesicles (SUVs), or large unilamellar vesicles (LUVs), with SUVs being preferred. The various liposome-nucleic acid complexes are prepared using methods well known in the art (Straubinger et. al., Methods of Immunology (1983), 101:512-527, which is herein incorporated by reference). For example, MLVs containing nucleic acid can be prepared by depositing a thin film of phospholipid on the walls of a glass tube and subsequently hydrating with a solution of the material to be encapsulated (U.S. Pat. No. 5,965,421, which disclosure is hereby incorporated by reference). Additionally, liposomes may be targeted to specific cell types by embedding a targeting moiety such as a member of a receptor—receptor ligand pair into the lipid envelope of the vesicle. Useful targeting moieties specifically bind cell surface ligands, for example, CD44v9 on the surface of a prostate cancer cell. Thus, in a further embodiment, the polynucleotide of the invention may be entrapped in a liposome and targeted to a cell expressing CD44v9 on its surface for treatment purposes

### EXAMPLE 1

### Expression of CD44 Variants in Prostate Cancer

RT-PCR was performed for amplification of most of the CD44 cDNA in 19 prostate cancer samples and 10 benign samples from prostate tissue not removed for cancer.

About 200 grams of prostatic tissue was sampled from fresh prostatectomy (grossly benign and malignant areas), suprapubic enucleation, or transurethral resection specimens at Gainesville Veterans Affairs Hospital with Internal Review Board approval and informed consent from patients. Tissue was frozen at −70° C. within 20 minutes after surgery. Presence or absence of carcinoma in tissue samples was assessed visually and confirmed by cryostat sectioning before further analysis and also verified on permanent, paraffin histology of adjacent areas. The percentage of epithelium was quantified in intervals of 5% for normalizing RNA band intensity on gel analysis.

Tissue was homogenized and total RNA was extracted with MidiPrep kits (Quiagen, Valencia, Calif.). RNA was precipitated with 1 volume of isopropanol and the RNA pellet was resuspended in 20 μl of diethylpyrocarbonate-treated water. RNA quality was verified by presence of 28S bands on 2.0% agarose gel electrophoresis and by measuring its concentration at wavelengths of 260 and 280 nm using a spectrometer. Yield of total RNA was 30-40 μg per sample. Complimentary DNA was synthesized from 2 μg of total RNA, in a 20 μL reaction using SuperScript II RNAse H-Reverse Transcriptase and the following reagents: oligo dT primer, dNTPs, and RNAse inhibitor (Invitrogen, Carlsbad, Calif.). Each RNA sample was heated to 75° C. for 5 minutes before cooling and adding reagents. The mixture, in a final volume of 20 μl, was incubated at 37° C. for 60 minutes, heated to 95° C. to denature the reverse transcriptase enzyme for 5 min, and then chilled on ice. Controls were also run with no reverse transcriptase in the mix. The cDNA concentration was determined by optical densitometry.

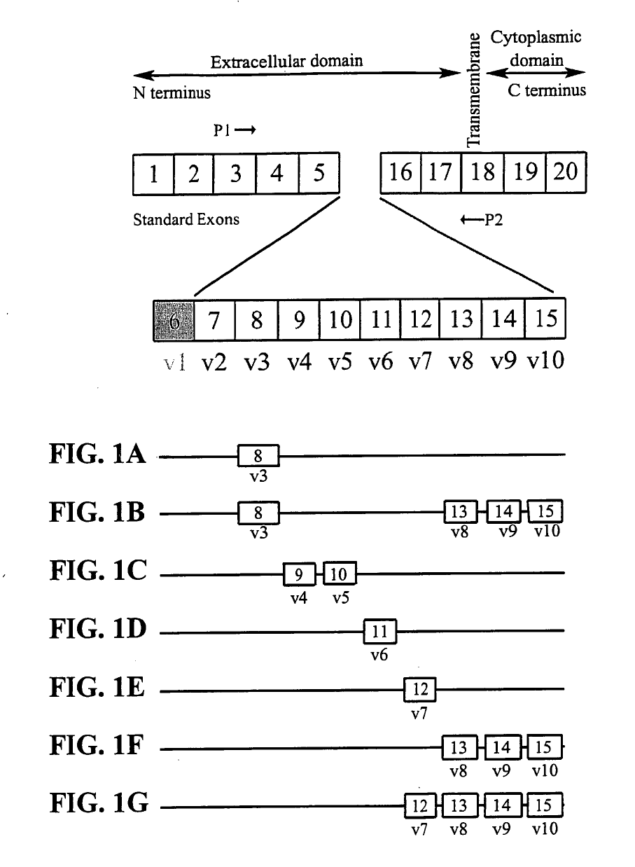

5 μL of CDNA was specifically amplified using primers P1 (SEQ ID NO: 1) and P2 (SEQ ID NO: 2) for the CD44 gene (FIG. 1). Our RT-PCR primer design and strategy used primer sequences we developed, similar to others reported in the literature for the CD44 gene, which could potentially amplify 1650 base pairs (bp) of CDNA. These primers flank the insertion site where variant exons 6-15 (v1-v10) can be inserted into the mRNA transcript. Sense primer P1 (SEQ ID NO: 1) has its origin 324 bp upstream from the insertion site, and antisense primer P2 (SEQ ID NO: 2) is 158 bp downstream. This sequence amplifies most of the CD44 cDNA (exons 3-18), including all variant exons (Tarin, D. and Matsumura, Y., 1993, Mod. Pathol. 11:80A).

Primers were designed to anneal to the CD44 sequence by rTth Taq polymerase (Applied Biosystems, Foster City, Calif.), an enzyme blend with both 5′→3′ polymerase and exonuclease activity, specifically designed for long fragment amplification. The enzyme has much higher fidelity than standard Taq polymerase and under specified reaction conditions, amplifies 22 kb of the human, β-globin gene and 42 kb of phage lambda DNA (Cheng, S et. al., 1994, Proc. Natl. Acad. Sci. USA 91:5695-5699). Thus, no “stuttering” occurs in transcription, as confirmed by the absence of dimers or trimers in our sequencing of bands.

The reaction volume was 50 μL, using the buffer conditions recommended by the manufacturer. Hot start amplification was performed for 30 cycles (94° C. for 1 minute, 56° C. for 1 minute and 72° C. for 2 minutes). A negative control (no template) and a positive control (containing primer specific for actin) were run with every sample batch. 1.2% agarose gel electrophoresis of PCR products was performed. The gel patterns were analyzed by routine ethidium bromide staining. Gel patterns were digitized (ImageJ 0.98t NIH Software) and number and intensity of molecular weight bands was recorded.

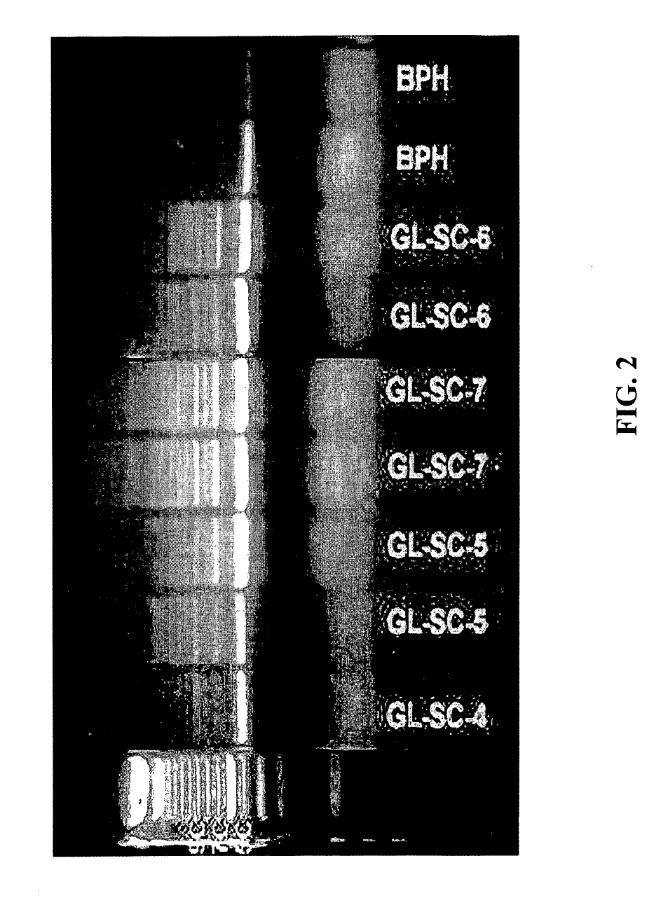

Agarose gel electrophoresis of RT-PCR products revealed that samples from the benign group (BPH) contained only a single bright band at 482 bp and faint bands at 600 bp (FIG. 2). In contrast to benign prostate, PC demonstrated overexpression of many long transcript CD44 mRNA bands. In all PC cases but one (18/19), bands representing longer mRNA were present from 700 to 1000 bp (FIG. 2).

To normalize for the heterogeneity of stroma in prostatectomy tissue, the intensities were divided by the percentage of epithelium, as determined by frozen section described above. By gel densitometry, we followed the example of CD44 expression studies in bladder cancer (Okamoto, I. et. al., 1998, J. Natl. Cancer Inst. 90:307-315; Miyake, H. et. al., 1998, Int. J. Cancer 18:560-564), extrahepatic bile duct carcinoma (Mikami, T. et. al., 2001, Am. J. Clin. Pathol. 116:369-376), and gastric carcinoma (Miwa, T. et. al., 1996, Cancer 77:25-29), and determined the CD44v/CD44s mRNA intensity ratio in cancer tissue and benign tissue (BPH). Use of this ratio takes into account any contribution of CD44s expressed by stromal cells. Inflammatory cells do not contain CD44v mRNA, as shown by in situ hybridization in colon cancer (Gorham, H. et. al., 1996, J. Clin. Pathol. 49:482-488). Prostate cancer tissue was compared only with benign tissue from benign specimens, avoiding any possible contamination of benign areas by CD44 variant isoforms shed by adjacent cancer.

By gel densitometry, CD44v/s ratio in tumor tissue was significantly higher than that in tissue from benign prostate with no tumor elsewhere in the gland (Table I). Ratio analysis confirmed consistently overamplified bands at 700 and 900 bp found in 18/19 cases of PC. All 10 benign control tissues lacked this pattern of expression. CD44v/s ratios of ≧0.39 were seen in PC bands of 700 bp and 900 bp. Generally, the complexity of gel patterns increased as Gleason score increased. In two score 7 tumors, 1000-1400 bp CD44 mRNA expression appeared, whereas in 5 score 4-6 tumors these bands were weak to absent. The 700-1400 bp CD44 mRNA expression intensities also seemed stronger in higher stage tumors (depending on extraprostatic extension, tumor size, and extent of lobes involved). In the two score 7 PC's, CD44v/s ratios were >0.51 for bands of 1000-1300 bp. These findings in our cases of PC prompted our subsequent studies of individual variants.

TABLE I
Ratio of CD44v/CD44s Bands after RT-PCR of Cancer and Benign Tissue

| | Cancer, | | | | | Cancer, | | | | |
|---|---|---|---|---|---|---|---|---|---|---|
| | Gleason score ≦6 | | | | | Gleason ≧7 | | | | |
| | and Stage T2 | | | | | and State T3a | | Benign | | |
| Band | Patients | | | | | Patients | | Patients | | |
| (bp length) | 1 | 2 | 3 | 4 | 5 | 1 | 2 | 1 | 2 | 3 |
| 482 | 1.00 | 1.00 | 1.00 | 1.00 | 1.00 | 1.00 | 1.00 | 1.00 | 1.00 | 1.00 |
| (CD44s) | | | | | | | | | | |
| 600 | 0.33 | 0.42 | 0.59 | 0.42 | 0.49 | 0.63 | 0.60 | 0.23 | 0.24 | 0.21 |
| 700 | 0.41 | 0.60 | 0.93 | 0.70 | 0.93 | 1.00 | 1.00 | 0.21 | 0.09 | 0.22 |
| 800 | 0.26 | 0.46 | 0.57 | 0.51 | 0.53 | 0.76 | 0.82 | 0.00 | 0.00 | 0.00 |
| 900 | 0.39 | 0.67 | 0.73 | 0.49 | 0.65 | 1.00 | 0.92 | 0.00 | 0.00 | 0.00 |
| 1000 | 0.24 | 0.39 | 0.51 | 0.37 | 0.51 | 0.74 | 0.69 | 0.00 | 0.00 | 0.00 |
| 1100 | 0.23 | 0.43 | 0.48 | 0.42 | 0.42 | 0.73 | 0.77 | 0.00 | 0.00 | 0.00 |
| 1200 | 0.00 | 0.00 | 0.00 | 0.30 | 0.30 | 0.67 | 0.63 | 0.00 | 0.00 | 0.00 |
| 1300 | 0.00 | 0.00 | 0.00 | 0.26 | 0.21 | 0.57 | 0.52 | 0.00 | 0.00 | 0.00 |

Ratios listed in bold are ratios ≧0.39 in bands of 900 base pairs and above.

### EXAMPLE 2

### Sequencing of CD44 Variants in Prostate Cancer

We cloned all of the mRNA transcripts from Example 1 that were between 500 and 1000 bp. Pooled RT-PCR samples were run on a 1.2% agarose gel for 4 hours. All bands were dissected from the gel, eluted, and rerun to confirm purity by molecular weight. The cDNAs from these bands were cloned into cut plasmid pGEMM-T (Promega, Madison, Wis.) and then ligated. Both sense and antisense strands were used for DNA sequencing. DNA sequencing was carried out on LI-COR model 4200IR series (Lincoln, Nebr.) using 1 μg of template and 2 pmol of primer. Comparison was made to the published sequence (Screaton, G. R. et. al., 1992, Proc. Natl. Acad. Sci. USA 89:12160-12164).

CD44 cDNA sequencing of 500-1000 bp bands revealed isoforms v7, v8, and v9 in 800, 900, and 1000 bp transcripts respectively. In addition, aberrant bands were found in the 600 and 700 bp transcripts with repeated sequences. The results of the sequencing of bands from 500-1000 bp were as follows: 500 bp, v10 (exons 3, 4, 5, 15, 16, 17); 600 bp v10 (exons 3, 4, 5, 15, 16, 17) and four repeated sequences; 700 bp, v10 (exons 3, 4, 5, 15, 16, 17) and one repeated sequence; 800 bp, v8, v9, v10 (exons 3, 4, 5, 13, 14, 15, 16, 17); 900 bp, v7, v8, v9, v10 (exons 3, 4, 5, 12, 13, 14, 15, 16), and 1000 bp, v7, v8, v9, v10 (exons 3, 4, 5, 12, 13, 14, 15, 16).

### EXAMPLE 3

### Expression and Sequencing of CD44v7-v10 in Prostate Cancer

The following study was prompted after finding expression of variant forms of CD44 in PC as described in the previous examples. RT-PCR was performed for amplification of exons 12-15 corresponding to v7-10 in malignant and benign prostate tissue.

Tissue was harvested from prostatectomy, enucleation or transurethral resection specimens, and in one case, a pelvic lymph node with metastatic tumor, within 20 minutes of surgery, with patient informed consent. Tissue was snap-frozen in liquid nitrogen and stored at −70° C. Frozen sections were performed on the tissue to verify that it either contained >50% tumor or was free of tumor.

Total RNA was isolated from the prostate tissues using Trizol (Invitrogen, Carlsbad, Calif.) as described by the manufacturer with some modifications. Briefly, 100 μg of frozen prostate tissue was ground on dry ice by cold mortar and pestle, and the powder was transferred directly into 2 ml of RTL lysis buffer (Qiagen, Valencia, Calif.) plus 20 μl of 2-mercaptoethanol (Sigma) and mixed by gentle inversion. To every 500 μl of this mixture, 1 ml of Trizol was added, mixed by inversion and incubated for 5 min at room temperature. This was followed by addition of 200 μl of chloroform, mixed again by inversion and incubated for 10 min at room temperature. The mixture was centrifuged at 14,000 rpm for 10 min at 4° C. in a bench centrifuge (Sorvall RMC 14). Total RNA was further purified by isopropanol precipitation. The isolated total RNA was dissolved in DEPC-treated water and quantified. The quality of RNA was verified by loading 6 μg of total RNA in separate wells, electrophoresis on 1% formaldehyde-agarose gel, and visualizing bands by ethidium bromide staining and UV transillumination.

For first-strand complementary DNA (cDNA) synthesis, 1 μg of total RNA was mixed with random hexamers (Invitrogen) to a final concentration of 50 ng per reaction and DEPC-treated water was used to bring the volume to 10 μ. The mixture was heated to 70° C. for 10 min and incubated on ice for 10 min. Reverse transcription was started by adding 10 μl of a master mix [4 μl of 5× buffer, 2 μl of 100 mM DTT, 1 μl of dNTPs, 1 μl (200 U) of SuperScript™ II RNaseH-Reverse Transcriptase (Invitrogen) and DEPC-treated water to a total volume of 10 μl]. A master mix of the above was prepared for 12 reactions and to each master mix 1 μl of RNAse Out (Invitrogen) was added. First strand cDNA synthesis was carried out at 25° C. for 10 min, followed by 42° C. for 45 min, and the reaction was stopped by heating at 70° C. for 3 min. The first-strand cDNAs were used as templates in PCR reactions.

For PCR amplification, 5 μl of the first-strand cDNA, 2.5 μl of 10 μM stock of the E3/P4 primer set (SEQ ID NOs: 3 and 4) (Sers, C. et. al., 1994, Cancer Res. 54:5689-5694) and 2.5 μl of Taq polymerase (diluted 1:10) were added to 37.5 μl of master mix to make a 50 μl reaction volume. Master mix consisted of 1× PCR buffer, 1.5 mM MgC12 and 200 μM dNTPs. The PCR conditions were as follows: 94° C. for 5 min; 5 cycles at 94° C. for 1 min, 50° C. for 1 min, and 72° C. for 3 min; 30 cycles at 94° C. for 1 min, 60° C. for 1 min, and 72° C. for 3 min; and a final extension of 72° C. for 10 min. Five μL of PCR product was loaded on a 0.8% agarose gel and electrophoresed at 100 volts for 1 hr and visualized by ethidium bromide staining and UV transillumination (FIG. 3).

Using the E3/P4 primer set (SEQ ID NOs: 3 and 4), a 608 kb band was amplified in 10 malignant, but no benign, prostate tissues tested and a 638 kb band was obtained in one case (FIG. 3A). Ribosomal 18S RNA served as the normalizer in each trial (FIG. 3B). The 608 kb band was also amplified in the lymph node metastasis of prostate cancer (data not shown).

The bands of interest were excised and the DNA extracted out of the gel using StrataPrep DNA Gel Extraction Kit (Stratagene, La Jolla, Calif.). The purified DNA was cloned into TOPO (TOPO TA cloning kit for sequencing, Invitogen) and the resulting plasmids were sequenced by the University of Florida DNA Sequencing Core. Sequences were compared to published ones (Screaton, G. R. et. al., 1992, Proc. Natl. Acad. Sci. USA 89:12160-12164) using JellyFish (LabVelocity, Burlingame, Calif.).

Purification, cloning into TOPO vector, and sequencing of the 608 and 638 kb bands showed that they contained the published human CD44v7-10 region as well as the preceding and following standard exons (FIG. 3C-D). Benign and tumor specimens yielded a 200 base pair band. Sequencing of this band revealed that it represented nonspecific binding of primer E3 to exon 5, with inclusion of CD44v10, and distal standard exons (FIG. 3E). The faint 100 base pair bands are primer-dimers.

### EXAMPLE 4

### In Situ Hybridization for CD44v7

Representative paraffin blocks were chosen from 12 consecutive and recent (less than 6 months) hospital cases of prostate cancer. Sense and antisense probes to CD44v7, both 32 base pairs long and biotinylated at the 5′ and 3′ ends, were synthesized by Invitrogen (Carlsbad, Calif.) based on the published exon sequence (Screaton, G. R. et. al., 1992, Proc. Natl. Acad. Sci. USA 89:12160-12164). The antisense probe is against the sequence in bold below (SEQ ID NO: 6), where capital letters indicate the exon and lower case letters indicate the flanking introns:

(SEQ ID NO: 5)

5′-actaatattgattccttcagATATGGACTCCAGTCATAGTACAACG
CTTCAGCCTACTGCAAATCCAAACACAGGTTTGGTGGAAGATTTGGACA
GGACAGGACCTCTTTCAATGACAACGCgtaagaat aacgatgctca
g-3′.

Probes were reconstituted in Tris-EDTA buffer, pH=8.0, to 1 nmol/μL. Paraffin sections were cut into a histobath with DEPC-treated water, and were air dried overnight. Slides were deparaffinized and rehydrated with xylene 2×5 minutes, 100% ethanol 2×2 minutes, 95% ethanol 2×2 minutes, and DEPC-water 3×1 minute. The tissue was digested by applying Proteinase K (DAKO, Carpinteria, Calif.) to the slide for 7 minutes at room temperature. The slides were rinsed in 3 changes of DEPC-water for 1 min. Hybridization solution consisted of 160 μL of denatured salmon sperm DNA added to 1.5 mL of the remaining components (40% deionized formamide, 10% dextran sulfate, 1× Denhardt's solution, 4×SSC, 1 mg/ml yeast t-RNA). Probe was diluted into hybridization solution to a final concentration of 100 nM, allowing 40 μL per slide. The probe was denatured by heating the hybridization solution at 75° C. for 5 minutes. This precluded secondary structure formation. The warm probe was applied to moist sections and coverslipped. The edges of coverslips were sealed with rubber cement. The slides were incubated at 37° C. in a moist chamber for 12 hours. Two stringency washes were done in 2×SSC with 0.1% Nonidet NP-40, at 37° C., for 5 minutes each. The slides were rinsed in phosphate buffered saline and peroxide block was performed with 3% H₂O₂ in methanol. Detection with tyramide signal amplification using the GenPoint kit (DAKO) was performed using the manufacturer's steps but not their stringency wash. Diaminobenzidine served as the chromogen. The slides were counterstained 1 minute with Mayer's hematoxylin. Staining was evaluated as: 1+: heterogeneous expression in 5-20% of cells; 2+: expression in 20-70% of cells; 3+: strong expression in over 70% of cells.

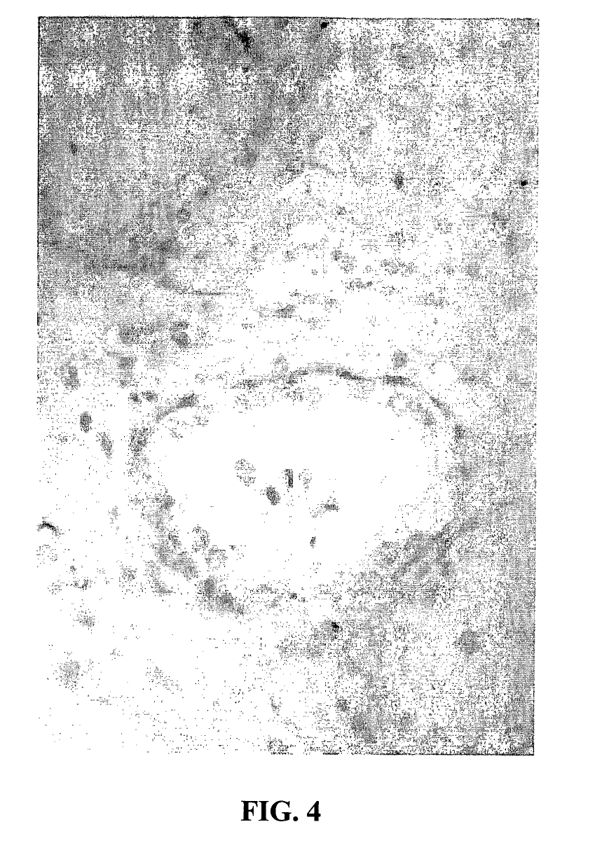

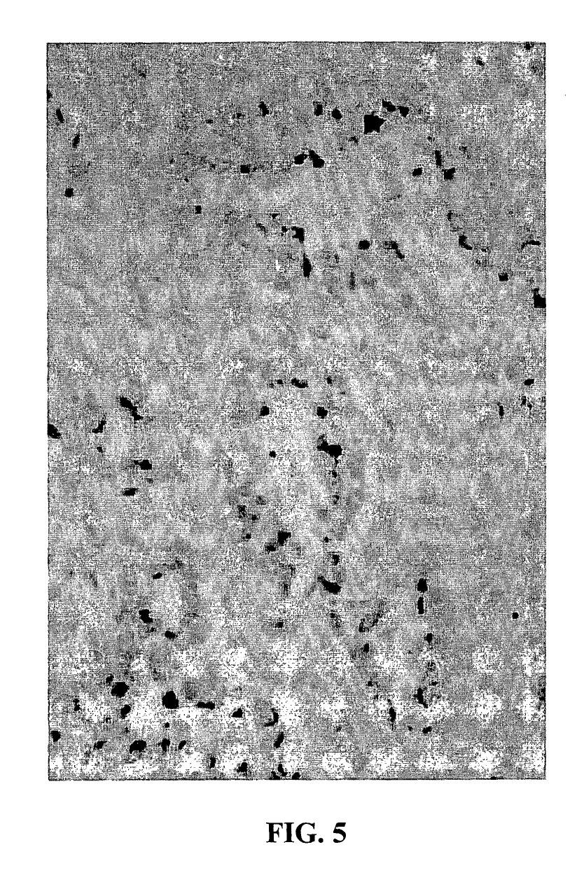

After hybridization with antisense probe, benign prostatic epithelium showed absent (or very rare) punctate cytoplasmic signal (FIG. 4). A signal, however, was noted in cancer of 11 of 13 cases, with nine of 13 having more than trace, focal reactivity (FIG. 5, Table II). Benign acini, in contrast, showed signal in 5 of 13 cases and two had more than trace reactivity. Signal in benign acini never exceeded that in cancer. High-grade prostatic intraepithelial neoplasia that accompanied cancer stained similar to cancer (FIG. 5). The strongest signal appeared to correlate with the presence of Gleason pattern 4, margin positivity (cases 4, 8, 9, 11), or tumor size greater than 2 cm (cases 3, 8, 9, 13). Also tested were two cases with seminal vesicle spread or lymph node metastasis from the past 3 years; both were from 2 year-old paraffin blocks and showed no signal. Hybridizations with sense probe consistently yielded no signal in cancer or benign glands.

TABLE II
In situ Hybridization of Prostate Cancer for CD44v7 (Exon 12)
(Mean Signal Intensity)

| | Gleason | | | Extraprostatic | Cancer | Benign acini |
|---|---|---|---|---|---|---|
| Case | score | Size (cm) | Margin | extension | signal | signal |
| 1 | 2 + 3 | 1 | neg | neg | 1+ | neg |
| 2 | 3 + 3 | 1 | neg | neg | 1+ | neg |
| 3* | 3 + 4 | 4 | neg | neg | 3+ | trace+ |
| 4 | 3 + 4 | 2 | pos | neg | 1+ | trace+ |
| 5 | 3 + 4 | 1.4 | neg | neg | trace+ | neg |
| 6 | 3 + 4 | 1.4 | neg | neg | 2+ | 1+ |
| 7 | 4 + 3 | 1 | neg | neg | neg | neg |
| 8 | 4 + 3 | 2.3 | pos | neg | 2+ | neg |
| 9 | 4 + 3 | 3 | pos | neg | trace+ | neg |
| 10 | 4 + 4 | 1.7 | neg | neg | neg | neg |
| 11 | 4 + 4 | 0.5 | pos | neg | 3+ | 1+ |
| 12 | 4 + 4 | 1.4 | neg | neg | 1+ | neg |
| 13 | 4 + 5 | 2.5 | neg | neg | 2+ | trace+ |

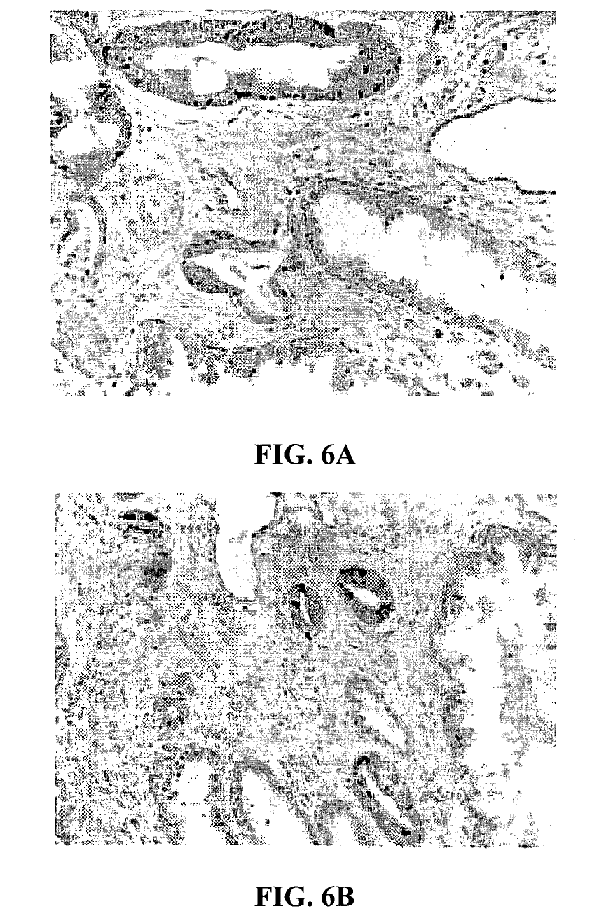

*Case pictured in FIG. 6A-B. Staining rated from 0+ to 4+.
Aggressive tumor features such as size >2 cm, extension to resection margins, or extraprostatic extension are in bold.

### EXAMPLE 5

### Immunohistochemistry for CD44v7/8

Two immunohistochemical studies were performed 6 years apart using the same antibody clone to CD44v7/8. Group 1 contained 26 consecutive recent prostatectomy cases and Group 2 contained 54 such cases. For analysis, tumor grades were compressed into four groups, based on the evidence that the percentage of Gleason grade 4 tumor at radical prostatectomy strongly predicts recurrence and progression (Sakr, W. A. et. al., 2000, Urology 56:730-734; Chan, T. Y. et. al., 2000, Urology 56:823-827).

Representative tissues were deparaffinized in xylene and alcohols and soaked 30 minutes in Tris-buffered saline, pH=7.5 with 0.1% Tween-20 (TBST). The slides were subject to steam heat antigen retrieval in citrate buffer, pH=6 for 2×30 minutes. The slides were quenched in 3% H₂O₂ in methanol for 10 minutes and rinsed well in distilled water, then in TBST. An “Inhibitor solution” (Ventana) was applied for 4 minutes followed by blocking antibodies for 10 minutes (20% normal swine serum in Tris-HCl, pH 7.6). The sections were immunostained with a 1:100 dilution of VFF9 antibody (Group 1 from Bender BioMed, Vienna; Group 2 from SeroTec, Raleigh, N.C.). This antibody is against a bacterial fusion protein. By Western blot it recognizes a 178-base pair sequence including all of the 103 bases of v7 and extending into v8 (Koopman, G. et. al., 1993, J. Exp. Med. 177:897-904). Biotin block (Ventana) was applied 3 minutes. A secondary antibody (Dako LSAB Kit mouse/goat/rabbit) was applied for 25 minutes. The slides were rinsed in TBST, covered with avidin-biotin complex 25 minutes, and rinsed in TBST. Color development was performed using diaminobenzidine, and the slides were counterstained with hematoxylin. Normal skin served as the positive control (Tran, T. A. et. al., 1997, Hum. Pathol. 28:809-814) and omission of primary antibody provided negative controls.

Cytoplasmic staining was quantified by 2 observers (KAI, CGP). The percent and intensity (0-4+) of epithelial cell staining in tumor, PIN, or benign categories were compared by paired t-tests on a per-case basis. Percent and intensity of tumor staining were correlated with grade by Fisher's exact test; with pT stage by Spearman rank correlation coefficient; and with margin status by Kendall's tau-b.

Analysis of experiments using the anti-CD44v7/8 antibody showed a mean 91% of cells stained in cancer, higher than the 56% in benign acini (p=0.0001), and with stronger intensity (2.56+ vs. 1.73+, p=0.0001). The percent of cells and intensity of staining in high-grade PIN were also both greater than in benign acini (both p<0.0001) (Table III, FIG. 6A-B). The intensity of staining increased with Gleason score (according to the 4 score groups) in Group 1 (p=0.01) but this trend was not significant in Group 2 (p=0.8). Percentage and intensity of cancer staining did not correlate significantly with tumor stage (p=0.13 and 0.17), margin status (p=0.20 and 0.23), or PSA failure (p=0.9 and 0.9). The staining intensity of intraprostatic tumor did not differ from that of tumor in the seminal vesicles (p=0.49) or lymph nodes (p=0.20). Basal cell hyperplasia showed greater percent (92%, p<0.0001) and intensity (2.39+, p<0.0001) of staining than non-hyperplastic benign acini. Lymphocytes and stroma were negative.

TABLE III
Immunohistochemical Staining for CD44 variant 7/8 from 80 Prostate Cancer Cases
Group 1

| | | Cancer | High grade | Benign | Seminal vesicle | | Metastases in | |
|---|---|---|---|---|---|---|---|---|
| | Gleason | acini | PIN | acini | invasive cells | | lymph nodes | |
| Cases | score | Strength** | Strength | Strength | N = | Strength | N = | Strength |
| 13 | 3-6 | 1.8 | 1.8 | 1.8 | 2 | 2.0 | 1 | 1 |
| 2 | 3 + 4 = 7 | 2.0 | 2.0 | 1.7 | 2 | 1.5 | 1 | 1 |
| 3 | 4 + 3 = 7 | 3.3 | 3.3 | 2.6 | 1 | 4.0 | 2 | 2.5 |
| 8 | 8-10 | 3.0 | 3.1 | 1.6 | 3 | 2.7 | 2 | 2.5 |
| Total 26 | | 2.7* | 2.7 | 1.8* | 8 | 2.4 | 6 | 2.0 |

Group 2

| | | Cancer | High grade | Benign | | |
|---|---|---|---|---|---|---|
| | Gleason | acini | PIN | acini | Seminal vesicle | Metastases in |
| Cases | score | Strength** | Strength | Strength | invasive cells | lymph nodes |
| 25 | 3-6 | 2.5 | 2.6 | 1.8 | None | None |
| 13 | 3 + 4 = 7 | 2.5 | 2.4 | 1.8 | None | None |
| 5 | 4 + 3 = 7 | 2.6 | 2.4 | 1.5 | None | None |
| 11 | 8-10 | 2.8 | 2.6 | 2.1 | None | None |
| Total 54 | | 2.6* | 2.5 | 1.7* | | |

*p = 0.0001 for strength of immunostaining in cancer compared to benign acini in both groups.
**Strength of cancer immunostaining increased with Gleason score in Group 1 (p = 0.01) but not Group 2 (p = 0.8).
Percent of cells staining averaged 91% in cancer and 56% in benign glands (p = 0.0001). Percent of cancer staining correlated with Gleason score (p = 0.046).

### EXAMPLE 6

### Western blotting for CD44v6 and CD44v7/8

Immunoblots were performed of five prostate cancer tissue specimens and five benign specimens using four different CD44 monoclonal antibodies after gel electrophoresis.

Protein was isolated in RIPA buffer (Upstate Biotechnologies, Lake Placid, N.Y.) from five frozen section confirmed Gleason score ≧7 tumor specimens from prostatectomy, and five confirmed benign specimens from men free of prostate cancer. Protein concentration in the lysates was determined by the Lowry method. Thirty mg of protein per sample in Laemmli sample buffer was run under non-denaturing conditions (7 μL stacking buffer with 1 mg/dL bromphenol blue, 25 mL 4× Tris Cl and 20 mL glycerin), or denaturing conditions (the same stacking buffer with 4 g/dL SDS and 3 g/dL dithiothreitol to break the disulfide bonds). Biotinylated SDS-PAGE standards (Bio-Rad, Hercules, Calif.) were run in tandem. Vertical electrophoresis was carried out at 35 mAmp constant current for 1 hr using a 15% SDS (w/v) nongradient polyacrylamide gel.

Dry blotting to nitrocellulose membrane was performed at 250 mAmp for 1 hr on a Hoeffer dry blotter. The membrane was dried and blocked 2 hr with PBS+1% Tween-20+3% bovine serum albumin. Primary antibodies applied were 1:1 dilution of anti-CD44 standard (prediluted, Zymed, S. San Francisco, Calif.), 1:1000 dilutions of anti-CD44v6 or CD44 v7/8 (SeroTec, Raleigh, N.C.), or 1:1 dilution of supernatant containing anti-CD44 variant 9 mouse monoclonal antibody from cultured HB-258 hybridoma cells (American Type Culture Collection, Manassas, Va.). The membrane was reacted overnight at 4° C. with primary antibody in PBS+1% Tween-20+1% bovine serum albumin. Biotinylated hamster anti-mouse secondary antibody (PharMingen, San Diego, Calif.) was used at a 1:140 dilution for 1 hr, followed by streptavidin-horseradish peroxidase (Sigma, St. Louis, Mo.) at 1:1000 for 1 hour. The Opti-4CN development kit from Bio-Rad was used for development without the amplification function. Each blot was repeated at least once with consistent results. Controls consisted of blotted membranes hybridized with all of the above steps except omission of the primary antibody. Two bands from 130-200 kD corresponding to nonspecific staining were disclosed, out of range for most isoforms of interest in this study.

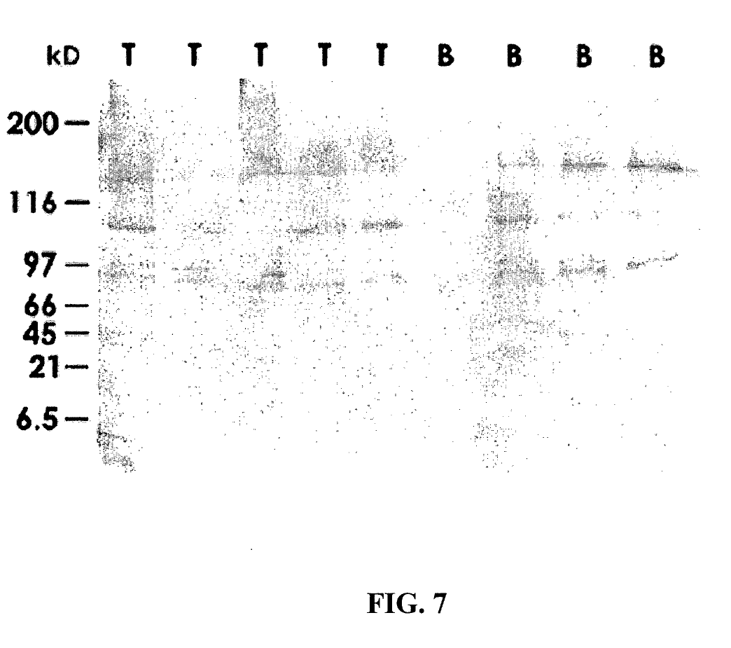

The non-denaturing condition best illustrated the differences between tumor and benign tissue. Immunoblotting with anti-CD44s (FIG. 7) revealed bands at 85 kD consistent with CD44 standard. Tumor samples showed less CD44s signal than most benign samples. Bands of reactivity at 45 kD were unique to tumor, and the fourth sample showed reactivity at 55 kD and 35 kD as well.

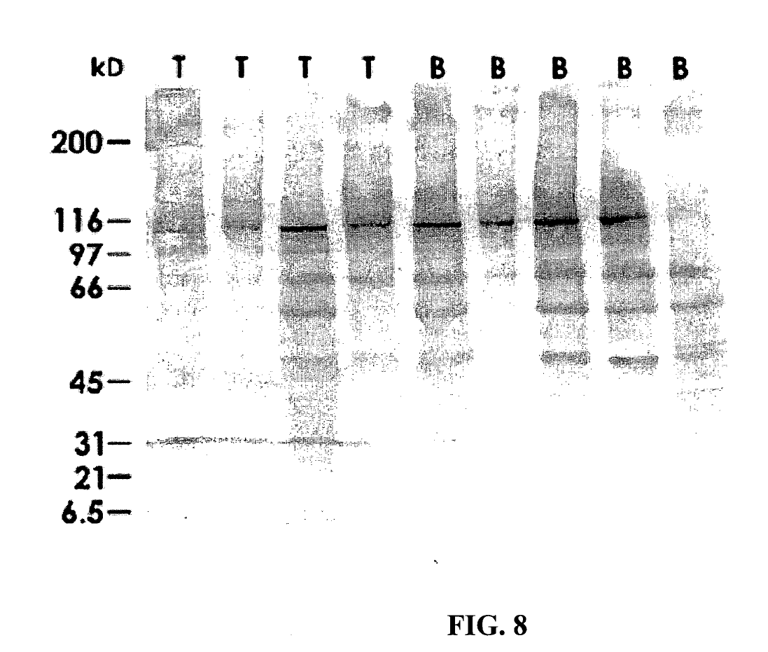

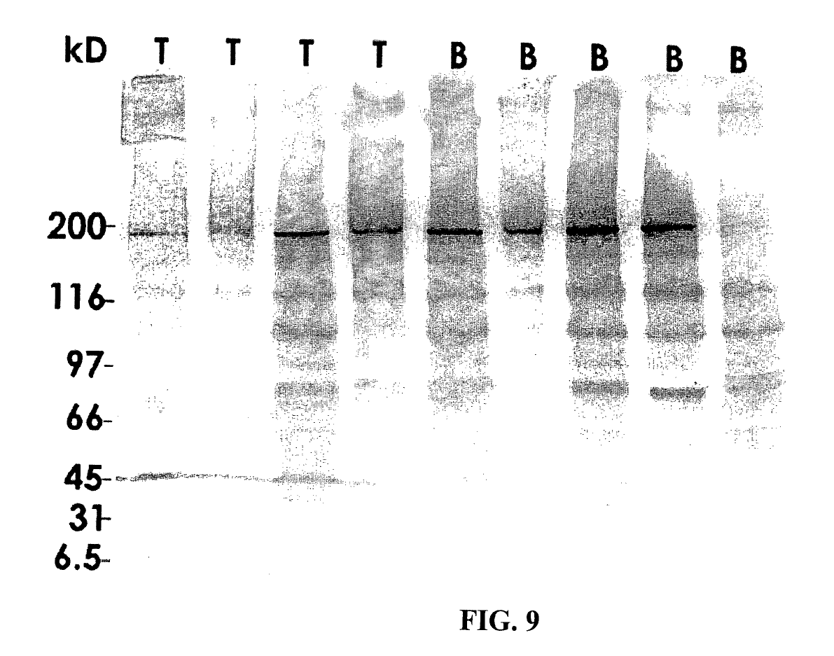

Blots hybridized with anti-CD44v6 (FIG. 8) showed that both tumor and benign specimens disclosed reactivity at 97 kD with no particular difference. For CD44v7/8 (FIG. 9), all samples disclosed major bands of reactivity at 97 kD, consistent with intact CD44v734 and often 75 kD with some reactivity in between these bands. In four tumor samples but not benign samples were prominent bands of reactivity at 45 kD. Tumor also appeared to have stronger low-molecular weight signal ranging from 21 kD down to 6.5 kD.

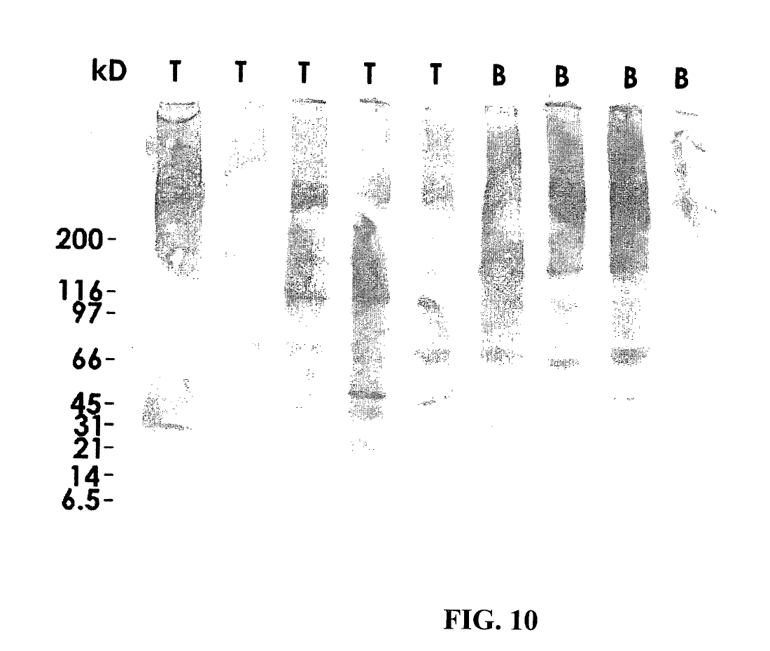

CD44v9 immunoblots had the most revealing pattern (FIG. 10). They showed overexpressed variant isoform at 116 kD in three of the five tumor samples, and prominent lower molecular weight bands of 14-66 kD. A prominent 55 kD band was seen particularly in the fourth and fifth tumor lanes. These lanes corresponded to patients with Gleason 3+4=7 tumors of particularly large volume with positive resection margins at prostatectomy, but with no seminal vesicle spread or metastasis. Benign samples did not show these lower molecular weight bands but its 85 kD bands, consistent with intact CD44s, were more prominent.

### EXAMPLE 7

### Immunohistochemistry for CD44v10

Immunohistochemical analysis of CD44v10 reactivity in 55 malignant and benign prostate cores and 18 benign tissues other than prostate was assessed.

The tissues were analyzed using Tissue Array Research Program tissue microarray slides (National Cancer Institute, Bethesda, Md.). Tissue sections were deparaffinized in xylene and alcohols and soaked 30 min in Tris-buffered saline, pH=7.5 with 0.1% Tween-20 (TBST). The slides were subject to steam heat antigen retrieval in citrate buffer, pH=6 for 2×30 minutes. The slides were then quenched in 3% H₂O₂ in methanol for 10 min and rinsed well in distilled water, then in TBST. An “Inhibitor solution” (Ventana) was applied for 4 min followed by blocking antibodies for 10 min (20% normal swine serum in Tris-HCl, pH 7.6). A 1:1000 dilution of monoclonal anti-CD44v10 (Biosource, Camarillo, Calif.) was applied at room temperature, overnight. Its specificity was confirmed by Western blot using normal lymph node as a positive control (Okamoto, I. et. al., 1998, J. Natl. Cancer Inst. 90:307-315). Biotin block (Ventana) was applied for 3 minutes followed by a secondary antibody (Dako LSAB Kit mouse/goat/rabbit) for 25 minutes. The slides were rinsed in TBST, covered with avidin-biotin complex for 25 minutes, and rinsed with TBST. Diaminobenzidine served as the chromogen, and slides were counterstained with hematoxylin. The negative control consisted of application of non-immune whole rabbit serum at 1:300 dilution. The number of benign and tumor tissues staining positive was compared by chi-square test. The two-tailed two-sample sign test was used to assess differences between staining of paired benign and cancer tissue.

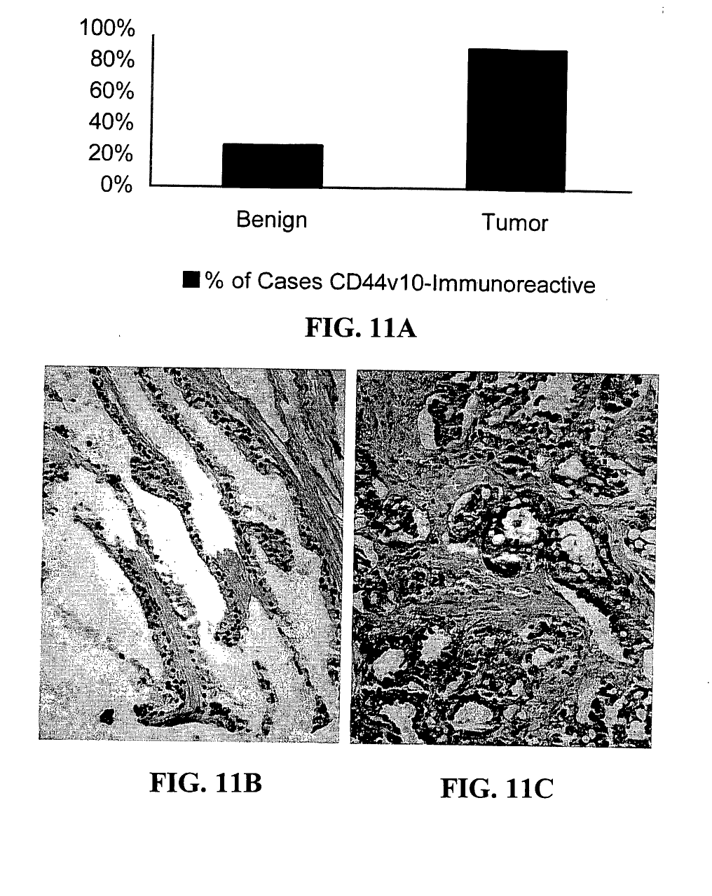

Immunoreactivity was present at 1+/3+in 10 out of 37 benign prostate (27%) samples, and at a mean 1.6+/3+in 49 of 55 tumor samples (89%) (p=0.001) (FIG. 11A). Reactivity for CD44v10 was cytoplasmic, with no nuclear staining and minimal to absent stromal background (FIG. 11C). In 23 of 27 cases in which paired benign and tumor tissue cores were provided and were evaluable, tumor tissue showed increased CD44v10 expression (p=0.01). No significant difference was noted in CD44v10 expression between lower grade (Gleason ≦6) and higher grade (Gleason ≧7) tumors (chi square test).

### EXAMPLE 8

### RNA Interference to Suppress Expression of CD44v9 and Muc18

We used RNAi to study the function of CD44 and Muc18 genes in prostate cancer cells.

The sequence of CD44v9 was found in GenBank and in Screaton, G. R. et. al. (1992, Proc. Natl. Acad. Sci. USA 89:12160-12164). The sequence of Muc18 was likewise found in published data (Wu, G-J. et. al., 2001, Gene, 279:17-31). Our strategy was to design and synthesize two pairs of 21 bp oligonucleotides for each molecule (two sense and two antisense) based on 21-nucleotide DNA fragments of each molecule (FIG. 12). The first 21 bp oligonucleotide, 1a, started in an area with 3G's but we used only GG in the oligonucleotide because another G is provided by the vector (after Apa I digestion and Klenow treatment). The GGG serves as the initiation site for the transcription of RNA Polymerase III (Pol III). The complementary strand of oligonucleotide la was synthesized as oligonucleotide 1b. To the upper strand of the second pair of 21 bp oligonucleotides, 2a, after CCC, we added TTTTT to give a Pol III transcription termination site. This was followed by an EcoRi site. The complementary strand of 2a was synthesized as oligonucleotide 2b. We used Hind III to make a connection between the two pairs of oligonucleotides.

The complementary oligonucleotides were annealed by mixing (from 250 pmol/ml stock) 10 μl of one oligonucleotide with 10 μl of the second oligonucleotide in 180 μl of water, boiling the 200 μl mixture in a 700 ml water bath for 5 minutes and gradual cooling of the water bath with the 200 μl mixture for 1 hour followed by incubation on ice for 10 minutes. Using a two-step cloning strategy, the two pairs of the 21-DNA nucleotide fragments were arranged head to head and sandwiched with a loop of 6 nucleotides (Hind III site) into plasmid vector U6pBS. Once the two pairs of the 21-DNA nucleotides were cloned in vector U6pBS, clean endotoxin-free plasmids were prepared using Endo Free Plasmid Maxi Kit (Qiagen) and the plasmids were then used in transfection experiments.

The goal was to generate short hairpin RNAs in transfected cells, to suppress specific CD44v9 or Muc18 gene activity as described (Paddison, P. J. et. al., 2002, Genes and Development, 16:948-958). Either parental PC3M or Gₛα prostate cancer cells were transfected with an expression vector that caused RNA interference (RNAi).

PC3M cells were purchased (American Type Culture Collection, Manassas, Va.) and incubated in RPMI 1640 with L-glutamine, 10% fetal calf serum, and antibiotics at 37° C. in a 5% CO₂ incubator. Gₛα prostate cancer cells were maintained in complete medium (RPMI 1640 supplemented with L-glutamine, 5% fetal calf serum, 12% horse serum, 50 U/mL penicillin, 50 μg/mL streptomycin, 20 μg/mL amphotericin).

Transfection of cells with vector U6pBS having RNAi constructs was optimized using the lipid agent Metafectene (Biontex, Munich, Germany). In brief, cells were plated at a density of 5×10⁵ cells per well in 2 ml of RPMI medium with serum and antibiotics in a 6-well plate. Cells were then transfected 24 hours later with vector U6pBS containing RNAi construct-Metafectene complexes. To formulate RNAi construct-lipofection complexes, two tubes were filled with 100 μL of RPMI without serum or antibiotics. To one tube, 10 μL (10 μg) of PCI-neo-GFP plasmid (which produces green fluorescent protein) as a reporter was added, followed by 10 μL (4 μg) of RNAi construct (for CD44v9 or Muc18). To the second tube, 10 μL of Metafectene were added. The contents of the two tubes were mixed well, then allowed to stand 25 min to allow formation of lipofection agent-DNA complexes for transfection. The incubated complex was added dropwise to the cells in each well, from which the serum-containing medium had been removed. Four μg of green fluorescent protein (GFP) was co-transfected to monitor the extent of transfection. Transfection in serum-free medium proceeded for 6 hours.

Others have documented that the RNAi effect takes up to four days after transfection to become maximal due to depletion of previously synthesized protein and may persist up to 6 days post-transfection (Kapadia, S. B. et. al., 2003, Proc. Natl. Acad. Sci. USA 100:2014-2018). We tried post-transfection incubations of 1-6 days, and optimized the interval for abrogation of protein expression by Western blot at 90 hours, in serum-containing medium.

Cells were successfully sorted by FACS into GFP-positive and GFP-negative cell groups using a FACSVantage SE flow cytometer (BD Biosciences, San Diego, Calif.). Portions of these cell populations were analyzed separately by Western blotting.

A pellet from centrifuged cultured cells was homogenized in RIPA lysis buffer (Upstate Biologicals, Lake Placid, N.Y.) plus the protease inhibitors 2 μg/mL Pepstatin, 1.5 μg/mL Leupeptin and 1 mM PMSF. Cell debris was removed by centrifugation and the protein concentration was estimated by Lowry method. The protein suspension was treated with 100 μL of 10% SDS. Forty μg of sample per lane was electrophoresed.

One part of 5× sample buffer (formulated as described by BIO-RAD) was added to four parts of the solubilized proteins. The proteins were then denatured for 5 minutes in 100° C. boiling water bath and 10 to 20 μL was loaded into wells of a 4% stacking gel. SDS-PAGE was performed according to the Laemmli method (Laemmli, U. K., 1970, Nature, 227:680-685) using 12% polyacrylamide gels. Five μL of Rainbow protein marker (RPN 756 Amersham Pharmacia, Piscataway, N.J.) were also loaded on each gel. After 2-hr electrophoresis the protein was transferred to nitrocellulose (Trans-Blot Transfer Medium, BIO-RAD) by dry blotting overnight. For Muc18 protein, a primary goat polyclonal IgG antibody (Santa Cruz Biotechnology, Santa Cruz, Calif.) was hybridized at 1:1000 dilution. Bovine anti-goat IgG labeled with horseradish peroxidase (Santa Cruz) was used as a secondary antibody at 1:10,000 dilution. To assess CD44v9, the membrane was reacted with supernatant from the hybridoma cell line HB-258 (ATCC, Manassas, Va.) which produces antibody against CD44v9, and this was used neat. Goat anti-mouse IgG labeled with horseradish peroxidase (Pierce, Rockford, Ill.) was used as a secondary antibody at 1:50,000 dilution. Reactivity was detected using a chemiluminescent system (SuperSignal West Pico Substrate, Pierce). Each experimental run was done twice.

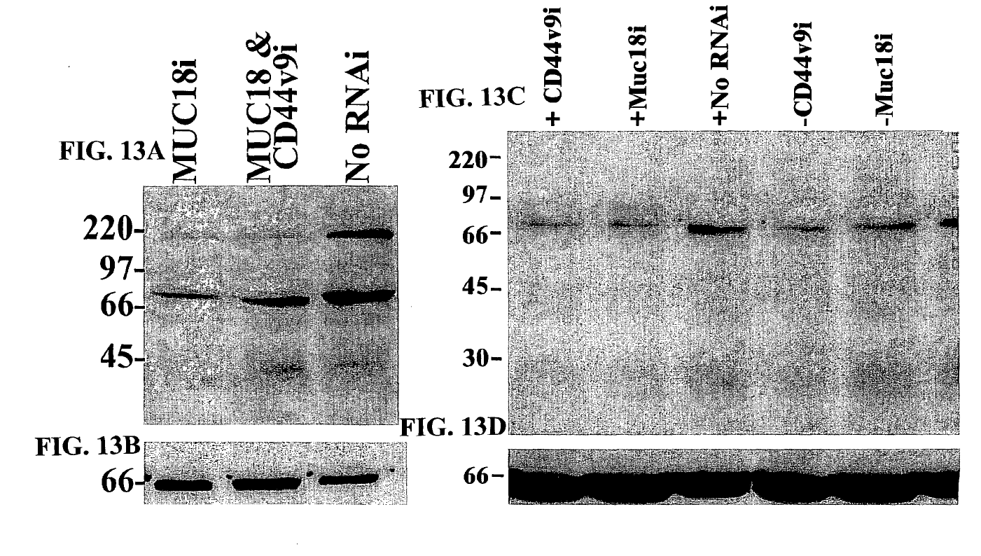

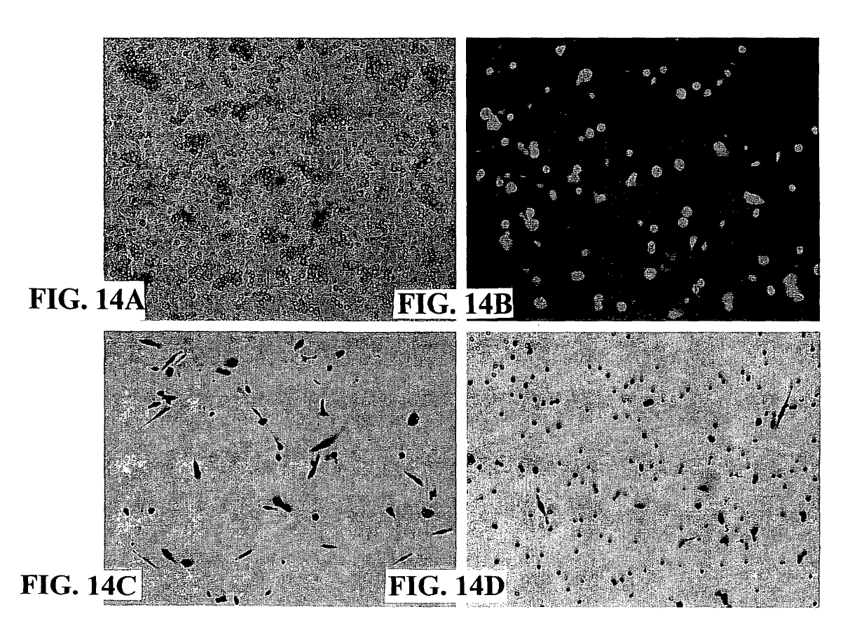

Two observations emerged. First, untreated cell lines differed in CD44v9 expression, with Gₛα cells tending toward stronger expression than PC3M cells. Second, Muc18 expression was decreased after RNAi for Muc 18, by Western blot analysis (FIG. 13A-B). Likewise, CD44v9-containing protein expression was decreased after RNAi for CD44v9 (FIG. 13C-D).

After FACS cell sorting, for the Gₛα cells, transfection of GFP plasmid alone yielded 46.9% GFP-positive cells; co-transfection with CD44v9 expression vector yielded 45.9% GFP-positive cells, and co-transfection of Muc18 expression vector yielded 34.1% GFP-positive cells. PC3M cell viability after FACS, however, was insufficient for further study to compare the GFP-positive and negative cells. Counts of live and dead cells showed similar survival rates of 54-63% for RNAi-treated PC3M cells and 73-76% for the Gscc cells. The GFP-positive cell population was shown by Western blot to have undergone successful RNA interference for the desired gene product (FIG. 13C-D). The percent of invaded cells that had been counted on the membrane as GFP-positive was 12.5-38.7% for the PC3M cells and 5.0-11.7% for the Gₛα cells, indicating that most of the cells that had invaded were untransfected.

### EXAMPLE 9

### RNA Interference Targeted to CD44v9 and Muc 18 to Reduce Invasion Prostate Cancer Cells

After PC3M and Gₛα cells were transfected with RNAi targeted against CD44v9 and Muc 18, we studied their invasion into Matrigel, a basement membrane-like substance.

Cells were transfected with RNAi and successfully sorted by FACS into GFP-positive and GFP-negative. cell groups using a FACSVantage SE flow cytometer (BD Biosciences, San Diego, Calif.). Portions of these cell populations were used in Matrigel invasion assays. Since we noted no change in protein expression by Western blot 24-48 hours following transfection, we waited 90 hours before performing the Matrigel invasion assay.

The experiment was performed in six-well Matrigel two-tier invasion chambers (Collaborative Biomedical Products, Bedford, Mass.), using a protocol similar to that used successfully by others (Sers, C. et. al., 1994, Cancer Res., 54:5689-5694). Prostate cancer cells (2.5×10⁵ cells per well expressing hairpin double-stranded CD44v interfering RNA or controls) were seeded in the upper insert in a serum-free basal medium (RPMI 1640 medium containing 0.1% BSA, 150 μg/ml of G418, 4 mM L-glutamine, 100 μg/ml penicillin G and 100 μg/ml streptomycin). The lower chamber contained chemoattractant medium consisting of 70% complete medium, 10% fetal bovine serum, and 20% conditioned medium obtained from subconfluent cultures. The incubations were carried out for 36 hrs.

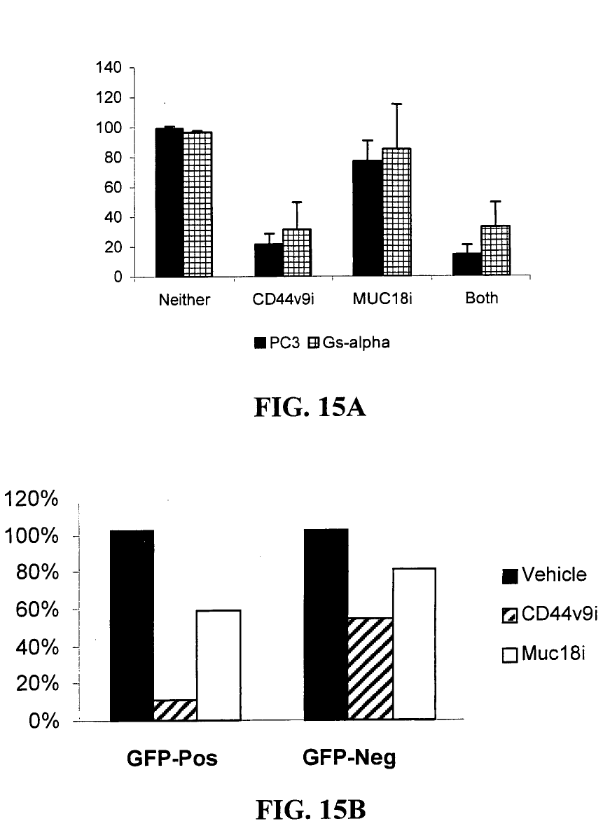

After this period, upper inserts were removed, and residual cells were removed from the upper Matrigel surface using cotton swabs. The invasive cells would penetrate through the Matrigel layer and would be on the outside bottom of the upper insert. While the membranes were still wet in culture dishes, the GFP-positive cells on the entire membrane were counted under fluorescent illumination. Gels were fixed, stained using Diff Quik staining (Dade Diagnostic, Aguar, PR), and mounted on glass slides. The total number of cells on the entire gel was counted. Photomicrographs were taken of Gₛα cells expressing GFP and RNAi targeted to CD44v9 and Matrigel following invasion by untreated Gₛα cells and Gₛα cells expressing RNAi targeted to CD44v9 (FIG. 14A-D).

The data from invasion assay were corrected for cell growth during experimental periods as follows: the experimental cells were plated at a density of 10⁵ cells per well in six well control inserts in chemoattractant medium and increase in cell number were determined after 48 hrs. Four experiments were done with PC3M cells and five were done with Gₛα cells. The results were expressed as mean±standard deviation.

The Percent Invasion is defined as 100× (Number of cells invading through entire Matrigel insert membrane)/Number of cells invading through entire control insert membrane. The Invasion Index is defined as 100× (Percent Invasion of treated cells)/(Percent Invasion of untreated cells). Significance of differences in Percent Invasion according to treatment (or transfection of Metafectene vehicle alone), were assessed by Student t-test.

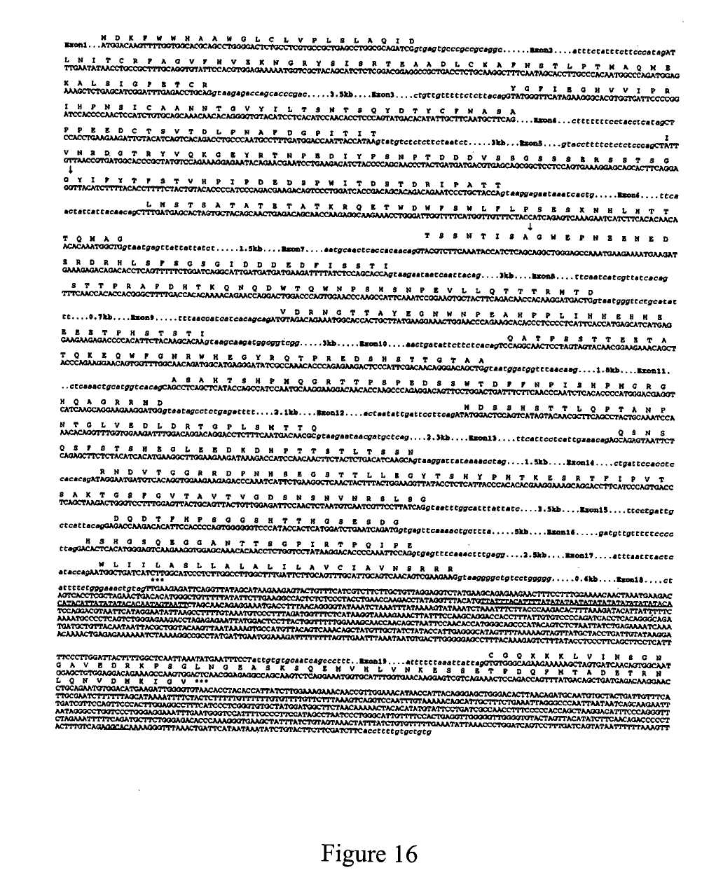

In four experiments with the PC3M cells and five with the Gₛα cells, markedly fewer cells receiving RNAi for CD44v9, or for CD44v9 plus Muc18, were able to invade through the membrane compared to the cells receiving no RNAi. Cells receiving RNAi for only Muc18 showed slightly inhibited invasion. The bar graph (FIG. 15A) shows mean differences in Invasion Index depending on transfection treatment of the cells. With CD44v9 RNAi, PC3M cells had an Invasion Index of 21.61%±7.03% of the invasive potential of control cells transfected with vehicle alone (p<0.001). Under this condition, the percent of the Gₛα cells invading was 31.28%±18.25% (p<0.001). Cells undergoing Muc18 inhibition had a higher Invasion Index of 76.9%±13.5% for the PC3M cells (p<0.05) and 84.8%±29.9% for the Gₛα cells versus controls (p=0.18), implying a stronger role for CD44v9 than Muc18 expression in allowing invasion. RNAi performed for both CD44v9 and Muc18 in both cell lines gave similar results to RNAi for CD44v9 only (both p<0.001).

Matrigel invasion assay performed after FACS cell sorting of Gₛα cells according to GFP positivity revealed differences in number of invaded GFP-positive and GFP-negative cells (FIG. 15B). Cells after CD44v9 interference had 11% invasion of GFP-positive cells versus 54% invasion of GFP-negative cells (p<0.005). Cells after Muc18 interference had 59% invasion of GFP-positive cells and 81% invasion of GFP-negative cells (p<0.025).

### EXAMPLE 10

### In Vivo RNA Interference Targeted to CD44v9

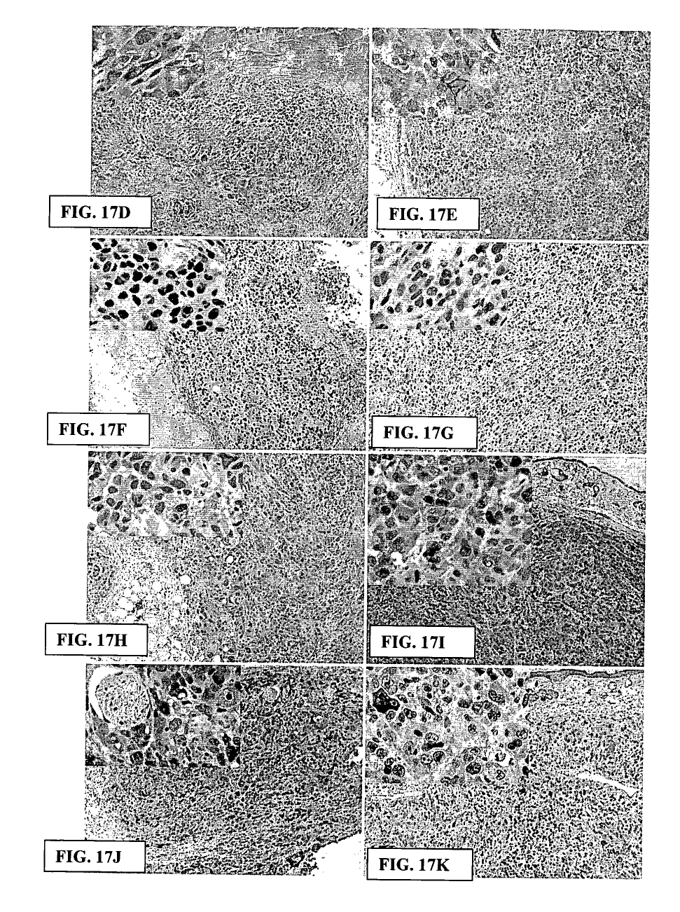

Confluent Gₛα-QL cells were trypsinized and counted. Four 7-wk-old mice (Jackson Laboratory, Bar Harbor, Me.) were injected in the right flank with 2×10⁶ tumor cells in 500 μL of medium. By 14 days after injection, a tumor mass was palpable and at 28 days, RNAi therapy commenced. This approach involved mixing volume of DNA containing 10 μg of pTracer plasmid having the RNAi construct with 10 μl of Metafectene before injection into the tumor. Both Metafectene and DNA were diluted separately in 100 μl of sterile serum-free RPMI before mixing. The mixture was then allowed to stand for 30 minutes before injection to allow the formation of Metafectene:DNA complexes. Intra-tumoral injection was performed five times weekly, angling the needle at different areas of the tumor during a given injection as well as varying the injection site for each injection occasion. Control animals were injected with 100 μL consisting of 50 μL medium plus 50 μL of Metafectene. After 11 days of injection, tumor volume decreased more than 50% in the treated animal but not in controls (FIGS. 17A-C), ruling out the possibility that the injections themselves could alter tumor size. The tumor's biologic potential was decreased by treatment, as shown by immunostains revealing decreased CD44v9 reactivity, as well as decreased proliferation, decreased angiogenic stimulation, and increased proapoptotic activity (FIGS. 17D-K).

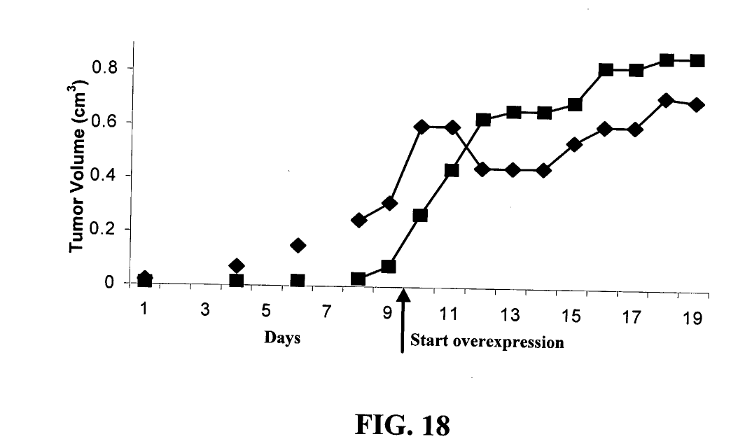

We chose the pTracer vector which has the GFP as well as a gene coding for blasticidin resistance to overexpress CD44 standard (CD44s). pTracer+CD44 standard in the correct orientation were used to transfect PC-3 cells. Pure populations were selected by blasticidin drug and by FACS cell sorting, after which the pure populations are being cultured in the face of blasticidin. The preferentially spliced CD44s was amplified by PCR from first strand cDNA made from total RNA extracted from benign prostate tissues. CD44 standard RNA was amplified from total RNA. Two PCR primers were designed: at the 5′ end: the XbaICD44 forward contains the Kozak translation initiation sequence with an ATG start codon for proper initiation of translation To this primer we have genetically engineered an Xba I restriction site before the Kozak translation initiation sequence. At the 3′ end, we also placed an Xba I restriction site into the XbaICD44 reverse after the stop codon TAA. We have successfully used these primers in PCR reactions to amplify full length CD44 using the CD44s as a template. PCR products were fractionated by electrophoresis, excised and eluted from the gel, and then cloned into TOPO. The TOPO reaction was used to transform Stbl2 E. coli cells (Invitrogen, Carlsbad, Calif.) which can faithfully replicate long plasmid DNA sequences such as ours. The E. coli cells that contained plasmids were selected for ampicillin resistance on LB agar plates. Single colonies of E. coli were picked and grown overnight in a shaking incubator. Plasmids were isolated and clones which appear to have CD44s were subjected to restriction analysis using ECoRV [EcoRV is in pTracer and full length CD44]. A single clone having CD44s in the correct orientation was identified by restriction analysis and confirmed by sequencing. Data indicate that tumor invasion of Gs-alpha and PC-3 cells is suppressed by overexpression of CD44 standard. Additionally, when CD44 standard overexpression was induced in target cells, tumor growth arrest was observed (FIG. 18).

1-17. (canceled)

18. A method of treating prostate cancer comprising the administration, to an individual having prostate cancer, of a composition comprising a carrier and: 1) antisense DNA molecules comprising nucleic acid sequences complementary to SEQ ID NO: 15, 16, 17, 18, or 19; or 2) RNAi molecules comprising a first polynucleotide sequence linked to a second polynucleotide that is complementary to said first polynucleotide sequence and wherein said first polynucleotide sequence is:
a) a contiguous span of at least X consecutive nucleotides of SEQ ID NOs: 15, 16, 17, or 18 and X is an integer between 15 and the number of nucleotides encoding the exon of each respective SEQ ID NO:;
b) a contiguous span of at least X consecutive nucleotides of SEQ ID NO: 19 and X is an integer between 15 and 459;
c) a contiguous span of Y nucleotides of SEQ ID NOs: 15, 16, 17, 18, or 19, wherein Y is an integer selected from the group consisting of 15, 16, 17, 18, 19, 20, 21, 22, 23, 24, 25, 26, 27, 28, 29, 30, 31, 32, 33, 34, 35, 36, 37, 38, 39, 50, 41, 42, 43, 44, 45, 46, 47, 48, 49, and 50;
d) SEQ ID NO: 7; or
e) SEQ ID NO: 9.

19. The method according to claim 18, wherein said composition is introduced into a locus containing prostate carcinoma cells.

20. The method according to claim 18, wherein said RNAi is linked via a polynucleotide sequence.

21. The method according to claim 18, wherein said RNAi comprises SEQ ID NO: 7 linked to SEQ ID NO: 8 or SEQ ID NO: 9 linked to SEQ ID NO: 10.

22. A method of reducing the invasiveness of carcinoma cells expressing CD44v7-10 comprising the administration of a composition comprising antisense nucleic acids or RNAi to an individual.

23. The method according to claim 22, wherein said composition is introduced into a locus containing prostate carcinoma cells.

24. The method according to claim 22, wherein said RNAi is linked via a polynucleotide sequence.

25. A method of reducing the growth of a CD44v9 expressing tumor comprising inducing the overexpression of CD44 standard in the cells of said tumor.

26. An isolated polynucleotide comprising a first polynucleotide sequence linked to a second polynucleotide that is complementary to said first polynucleotide sequence and wherein said first polynucleotide sequence is:
a) a contiguous span of at least X consecutive nucleotides of SEQ ID NOs: 15, 16, 17, or 18 and X is an integer between 15 and the number of nucleotides encoding the exon of each respective SEQ ID NO:;
b) a contiguous span of at least X consecutive nucleotides of SEQ ID NO: 19 and X is an integer between 15 and 459;
c) a contiguous span of Y nucleotides of SEQ ID NOs: 15, 16, 17, 18, or 19, wherein Y is an integer selected from the group consisting of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50;
d) SEQ ID NO: 7;
e) SEQ ID NO: 9;
f) SEQ ID NO: 7 and said second polynucleotide sequence is SEQ ID NO: 8; or
g) SEQ ID NO: 9 and said second polynucleotide sequence is SEQ ID NO: 10.

27. A composition comprising a carrier and a polynucleotide according to claim 26.

28. A method of inducing apoptosis in a cell expressing a CD44 variant comprising contacting said cells with a composition comprising a carrier and: 1) antisense DNA molecules comprising nucleic acid sequences complementary to SEQ ID NO: 15, 16, 17, 18, or 19; or 2) RNAi molecules comprising a first polynucleotide sequence linked to a second polynucleotide that is complementary to said first polynucleotide sequence and wherein said first polynucleotide sequence is:
a) a contiguous span of at least X consecutive nucleotides of SEQ ID NOs: 15, 16, 17, or 18 and X is an integer between 15 and the number of nucleotides encoding the exon of each respective SEQ ID NO:;
b) a contiguous span of at least X consecutive nucleotides of SEQ ID NO: 19 and X is an integer between 15 and 459;
c) a contiguous span of Y nucleotides of SEQ ID NOs: 15, 16, 17, 18, or 19, wherein Y is an integer selected from the group consisting of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50;
d) SEQ ID NO: 7; or
e) SEQ ID NO: 9.

29. The method according to claim 28, wherein said composition is introduced into a loci containing said cells.

30. The method according to claim 29, wherein said composition is introduced via stereotactic or intratumoral injection.

31. A method of reducing the volume of a tumor comprising cells expressing a CD44 variant comprising contacting said tumor with a composition comprising a carrier and: 1) antisense DNA molecules comprising nucleic acid sequences complementary to SEQ ID NO: 15, 16, 17, 18, or 19; or 2) RNAi molecules comprising a first polynucleotide sequence linked to a second polynucleotide that is complementary to said first polynucleotide sequence and wherein said first polynucleotide sequence is:
a) a contiguous span of at least X consecutive nucleotides of SEQ ID NOs: 15, 16, 17, or 18 and X is an integer between 15 and the number of nucleotides encoding the exon of each respective SEQ ID NO:;
b) a contiguous span of at least X consecutive nucleotides of SEQ ID NO: 19 and X is an integer between 15 and 459;
c) a contiguous span of Y nucleotides of SEQ ID NOs: 15, 16, 17, 18, or 19, wherein Y is an integer selected from the group consisting of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50;
d) SEQ ID NO: 7; or
e) SEQ ID NO: 9.

32. The method according to claim 31, wherein said composition is introduced into a loci containing said cells.

33. The method according to claim 32, wherein said composition is introduced via stereotactic or intratumoral injection.

34. The method according to claim 33, wherein said stereotactic injection is MRI-guided.

35. A method of treating prostate cancer comprising the intratumoral or stereotactic injection of an antibody that specifically binds to a CD44 variant into an individual with prostate cancer.

36. The method according to claim 35, wherein said antibody specifically binds to CD44v9.


SEQUENCE LISTING (38 sequences)

SEQ ID NO: 1 - DNA, 27 bp, Artificial Sequence
  P1 oligonucleotide primer located in exon 1 of CD44, upstream to the CD44 variant exon region.
atggacaagttttggtggcacgcagcc

SEQ ID NO: 2 - DNA, 28 bp, Artificial Sequence
  P2 oligonucleotide primer located in exon 20 of CD44, upstream to the CD44 variant exon region.
ttacaccccaatcttcatgtccacattc

SEQ ID NO: 3 - DNA, 21 bp, Artificial Sequence
  E3 oligonucleotide primer located upstream to the CD44 variant exon region.
agcccagaggacagttcctgg

SEQ ID NO: 4 - DNA, 28 bp, Artificial Sequence
  P4 oligonucleotide primer located downstream to the CD44 variant exon region.
gatgccaagatgatcagccattctggaa

SEQ ID NO: 5 - DNA, 142 bp, Artificial Sequence
  Partial nucleotide sequence of the CD44v7 gene corresponding to intron 12, exon 12, and intron 13.
actaatattgattccttcagatatggactccagtcatagtacaacgcttcagcctactgcaaatccaaacacaggtttggtggaagatttggacaggacaggacctctttcaatgacaacgcgtaagaataacgatgctcag

SEQ ID NO: 6 - DNA, 32 bp, Artificial Sequence
  Portion of the nucleotide sequence encoding CD44v7 used to create sense and antisense probes for in situ hybridization experiments.
ttcagcctactgcaaatccaaacacaggtttg

SEQ ID NO: 7 - DNA, 21 bp, Artificial Sequence
  Oligonucleotide primer corresponding to Oligo 1a of Figure 12 used to create CD44v9 dsRNA.
ggtcctttggagttactgcaa

SEQ ID NO: 8 - DNA, 25 bp, Artificial Sequence
  Oligonucleotide primer corresponding to Oligo 1b of Figure 12 used to create CD44v9 dsRNA.
agctttgcagtaactccaaaggacc

SEQ ID NO: 9 - DNA, 26 bp, Artificial Sequence
  Oligonucleotide primer corresponding to Oligo 2a of Figure 12 used to create CD44v9 dsRNA.
agctttgcagtaactccaaaggaccc

SEQ ID NO: 10 - DNA, 22 bp, Artificial Sequence
  Oligonucleotide primer corresponding to Oligo 2b of Figure 12 used to create CD44v9 dsRNA.
gggtcctttggagttactgcaa

SEQ ID NO: 11 - DNA, 21 bp, Artificial Sequence
  Oligonucleotide primer corresponding to Oligo 1a of Figure 12 used to create Muc18 dsRNA.
ggcagcacagcccttctgaaa

SEQ ID NO: 12 - DNA, 25 bp, Artificial Sequence
  Oligonucleotide primer corresponding to Oligo 1b of Figure 12 used to create Muc18 dsRNA.
agcttttcagaagggctgtgctgcc

SEQ ID NO: 13 - DNA, 32 bp, Artificial Sequence
  Oligonucleotide primer corresponding to Oligo 2a of Figure 12 used to create Muc18 dsRNA.
agcttttcagaagggctgtgctgccctttttg

SEQ ID NO: 14 - DNA, 32 bp, Artificial Sequence
  Oligonucleotide primer corresponding to Oligo 2b used to create Muc18 dsRNA.
aattctttttgggcagcacagcccttctgaaa

SEQ ID NO: 15 - DNA, 142 bp, Homo sapiens; identical to SEQ ID NO: 5
  misc_feature (1)..(142): CD44 Exon 12 (142 Base Pairs) Accession # L05417

SEQ ID NO: 16 - DNA, 130 bp, Homo sapiens
  misc_feature (1)..(130): CD44 Exon 13 (130 Base Pairs) Accession # L05418
ttcattcctcattgaaacagagcagagtaattctcagagcttctctacatcacatgaaggcttggaagaagataaagaccatccaacaacttctactctgacatcaagcagtaaggattataaaacctag

SEQ ID NO: 17 - DNA, 244 bp, Homo sapiens
  misc_feature (1)..(244): CD44 Exon 14 (244 Base Pairs) Accession # L05419
ctgattccacctccacacagataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggtaatttggcatttattatc

SEQ ID NO: 18 - DNA, 103 bp, Homo sapiens
  misc_feature (1)..(103): CD44 Exon 15 (103 base pairs) Accession # L05420
ttcctgattgctcattacaggagaccaagacacattccaccccagtggggggtcccataccactcatggatctgaatcagatggtgagttcaaaactgcttta

SEQ ID NO: 19 - DNA, 459 bp, Homo sapiens
  misc_feature (1)..(459): CD44 Exons 12-15 (459 base pairs)
atatggactccagtcatagtacaacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatg

SEQ ID NO: 20 - DNA, 608 bp, Artificial Sequence
  Alternative sequence that can be used for the production of RNAi or antisense DNA for use in the methods of the subject invention.
agcccagaggacagttcctggatcaccgacagcacagacagaatccctgctaccaatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacattaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatc

SEQ ID NO: 21 - DNA, 206 bp, Homo sapiens
  misc_feature (1)..(206): NCBI Accession No. L05407
atttctatttcttcccatagatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtaagagaccagcacccgac

SEQ ID NO: 22 - DNA, 154 bp, Homo sapiens
  misc_feature (1)..(154): NCBI Accession No. L05408
ctgttgtttttctcttacaggtatgggttcatagaagggcacgtggtgattccccggatccaccccaactccatctgtgcagcaaacaacacaggggtgtacatcctcacatccaacacctcccagtatgacacatattgcttcaatgcttcag

SEQ ID NO: 23 - DNA, 109 bp, Homo sapiens
  misc_feature (1)..(109): NCBI Accession No. L05409
ctttttttcctacctcatagctccacctgaagaagattgtacatcagtcacagacctgcccaatgcctttgatggaccaattaccataagtatgtctctcttctaatct

SEQ ID NO: 24 - DNA, 271 bp, Homo sapiens
  misc_feature (1)..(271): NCBI Accession No. L05410
gtaccttttctctctcccagctattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagtaaggagaataaatcactg

SEQ ID NO: 25 - DNA, 169 bp, Homo sapiens
  misc_feature (1)..(169): NCBI Accession No. L05411
ttcaactattattacaacagctttgatgagcactagtgctacagcaactgagacagcaaccaagaggcaagaaacctgggattggttttcatggttgtttctaccatcagagtcaaagaatcatcttcacacaacaacacaaatggctggtaatgagttattattatct

SEQ ID NO: 26 - DNA, 166 bp, homo sapiens
  misc_feature (1)..(166): NCBI Accession No. L05412
aatgcaactcaccacaacaggtacgtcttcaaataccatctcagcaggctgggagccaaatgaagaaaatgaagatgaaagagacagacacctcagtttttctggatcaggcattgatgatgatgaagattttatctccagcaccagtaagaataatcaattacag

SEQ ID NO: 27 - DNA, 154 bp, Homo sapiens
  misc_feature (1)..(154): NCBI Accession No. L05413
ttcaatcatcgttatcacagtttcaaccacaccacgggcttttgaccacacaaaacagaaccaggactggacccagtggaacccaagccattcaaatccggaagtgctacttcagacaaccacaaggatgactggtaatgggttctgcatattt

SEQ ID NO: 28 - DNA, 157 bp, homo sapiens
  misc_feature (1)..(157): NCBI Accession No. L05414
tttaaccatcatcacagcagatgtagacagaaatggcaccactgcttatgaaggaaactggaacccagaagcacaccctcccctcattcaccatgagcatcatgaggaagaagagaccccacattctacaagcacaagtaagcaagatggcggtcgg

SEQ ID NO: 29 - DNA, 169 bp, Homo sapiens
  misc_feature (1)..(169): NCBI Accession No. L05415
aactgatattcttctcacagtccaggcaactcctagtagtacaacggaagaaacagctacccagaaggaacagtggtttggcaacagatggcatgagggatatcgccaaacacccagagaagactcccattcgacaacagggacagctggtaatggatggtttaacaag

SEQ ID NO: 30 - DNA, 172 bp, Homo sapiens
  misc_feature (1)..(172): NCBI Accession No. L05416
ctcaaactgcatggtcacagcagcctcagctcataccagccatccaatgcaaggaaggacaacaccaagcccagaggacagttcctggactgatttcttcaacccaatctcacaccccatgggacgaggtcatcaagcaggaagaaggatgggtaatagcctctgagatttt

SEQ ID NO: 31 - DNA, 142 bp, Homo sapiens; identical to SEQ ID NO: 5
  misc_feature (1)..(142): NCBI Accession No. L05417

SEQ ID NO: 32 - DNA, 130 bp, Homo sapiens; identical to SEQ ID NO: 16
  misc_feature (1)..(130): NCBI Accession No. L05418

SEQ ID NO: 33 - DNA, 244 bp, Homo sapiens; identical to SEQ ID NO: 17
  misc_feature (1)..(244): NCBI Accession No. L05419

SEQ ID NO: 34 - DNA, 103 bp, Homo sapiens; identical to SEQ ID NO: 18
  misc_feature (1)..(103): NCBI Accession No. L05420

SEQ ID NO: 35 - DNA, 112 bp, Homo sapiens
  misc_feature (1)..(112): NCBI Accession No. L05421
gatgttgtttttccccttaggacactcacatgggagtcaagaaggtggagcaaacacaacctctggtcctataaggacaccccaaattccaggtgagtttcaaactttgagg

SEQ ID NO: 36 - DNA, 119 bp, Homo sapiens
  misc_feature (1)..(119): NCBI Accession No. L05422
atttaatttactcataccagaatggctgatcatcttggcatccctcttggccttggctttgattcttgcagtttgcattgcagtcaacagtcgaagaaggtaaggggctgtcctggggg

SEQ ID NO: 37 - DNA, 989 bp, Homo sapiens
  misc_feature (1)..(989): NCBI Accession No. L05423
ctattttctgggaaactgtagttgaagagattcaggttatagcataagaagagtactgtttcatcgtcttcttgctgttaggaggtctatgaagcagagaagaactttcctttggaaaacaactaaatgaagacagtcacctcgctagaactgacacatgggctgtttttatattcttgaaggccactctctccctacctgaaccaagacctataggtttacatgttatttacattttatatataatatatatatatatatatacacatacattatatatacacaatagtaattctagcaacagaggaaatgacctttaacaggggtataaatctaaatttataaaagtataaatctaaatttcttacccaagacactttaaagatacattatttttctccaggacgtaattcataggaatattaagccttttgtaaatgtccctttagatggtttctcataaggtaaaagaaacttatttccaagcaggaccacctttattgtgtccccagatcacctcacagggcagaaaaatgcccctcagtctgggagaagacctagagagaattatggactccttactggtttttggaaagcaaccaacagctaattccaacaccatgggcagcccatacagtctctaattatctgagaaaatcaaatgatgctgttacaataattacgctggtacaagttaataaaagtgccatgttacagtcaaacagctatgttgctatctataccattgagggcatagttttaaaaagtagttatgctacctgattgtataaggaacaaaactgagagaaaaaatctaaaaggccgcctatgattgaatggaaagattttttttagttgaatttaaataatgtgacttgggggagcctttacaaagagtctttatacctcccttcagcttcctcattttcccttggattacttttggctcaattaaatatgaatttcctattgtgtgcaatcagcccttt

SEQ ID NO: 38 - DNA, 930 bp, Homo sapiens
  misc_feature (1)..(930): NCBI Accession No. L05424
attttttaaattattaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgttttccactgaggttgggggttggggtgtactagttacatatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtg